(19) United States
(12) Patent Application Publication
(10) Pub. No.: US 2010/0136598 A1
(43) Pub. Date: Jun. 3, 2010

(54) NOVEL MESENCHYMAL PROGENITOR CELLS DERIVED FROM HUMAN BLASTOCYST-DERIVED STEM CELLS

(76) Inventors: Raimund Strehl, Goteborg (SE); Katarina Emanuelsson, Goteborg (SE); Fredrik Wessberg, Goteborg (SE); Sven Johan Hyllner, MoIndal (SE)

Correspondence Address: BUCHANAN, INGERSOLL & ROONEY PC, POST OFFICE BOX 1404, ALEXANDRIA, VA, 22313-1404, US

(21) Appl. No.: 12/522,894

(22) PCT Filed: Jan. 11, 2008
(86) PCT No.: PCT/EP2008/000190

§ 371 Date: Nov. 20, 2009

Related U.S. Application Data
(60) Provisional application No. 60/879,799, filed on Jan. 11, 2007.

(51) Int. Cl. C12Q 1/02; C12N 5/071; C12N 5/02; C12P 1/00

(52) U.S. Cl. 435/29; 435/366; 435/395; 435/41

(57) ABSTRACT

Described herein is a mesenchymal human progenitor (hBS-MP) cell population derived from human blastocyst-derived stem (hBS) cells and a method to obtain the progenitor cell population in which is eliminated the need of co-culture steps, cell sorting, manual selection, and transfections. Also, the use of the hBS-MP cells in drug discovery and specifically for toxicity testings as well as for therapeutic use is made possible.

### BACKGROUND OF THE INVENTION

The successful isolation of human embryonic stem (hES) cells, or as used herein, hBS cells (human blastocyst-derived stem cells, (Thomson et al., 1998) started a new field of research and has raised expectations that hBS cells may provide a unique source of functional human cells for future cell therapy and tissue engineering as well as for applications in the drug discovery process or for toxicity detections and measurements of potential drug candidates as well as chemicals in our every-day environment. Since hBS cell lines can be expanded in vitro without an apparent limit and have the potential to differentiate into derivatives of all three embryonic germ layers (pluripotency) they are a potentially valuable source of cells where autologous cells can not be used. Progress has been made in directing hBS cell differentiation towards specific cell types such as cardiomyocytes, neural, hepatocytes or connective tissue cells (Sottile et al., 2003) in the meantime. However, the direct derivation of pure populations of functionally differentiated cells from hBS cells still poses a challenge and there is a significant risk that the transplantation of undifferentiated hBS cells may lead to tumor formation in the recipients.

Progenitor cells are immature cells in an intermediate stage of development, i.e. between stem cells and fully mature cells. Like stem cells, progenitor cells have a capacity for self-renewal and differentiation, but these properties are more limited i.e. progenitor cells have a more finite lifespan and give rise to a more lineage restricted progeny (mulitpotency).

From a safety aspect the application of progenitor cells instead of undifferentiated hBS cells for therapeutic purposes is favorable since the differentiation pathway is already partly determined and the risk of tumor formation should therefore be reduced. From a technical point of view progenitor cultures are expected to be more stable and easier to scale up in vitro than undifferentiated hBS cells which would provide an advantage for bulk production of cells for therapy as well as provide large amounts of homogeneous cells for assay applications.

Research to develop germ layer specific progenitor cells from hBS cells has been reported before. With respect to the mesoderm, progenitor cell lines have been derived from hBS cells by transfection of embryoid body derived cells with human telomerase reverse transcriptase (Xu et al., 2004), by co-culture with mouse OP9 cells (Barberi et al., 2005), by fluorescence activated cell sorting (Lian et al., 2006) or by manual selection of cell populations (Stojkovic et al., 2005; Olivier et al., 2006).

We herein present a novel mesenchymal progenitor (MP) cell type from hBS cells, the hBS-MP and protocols allowing simple and reproducible derivation of such hBS-MP cell lines from undifferentiated hBS cells by repetitive enzymatic passaging. In contrast to other methods, the protocols presented here do not require embryoid body formation, cell transfection, co-culture, cell sorting or subjective manual selection of certain cell types, the latter normally involving for instance a step of visual inspection and directed mechanical and/or enzymatic detachment of desired cell types. In addition, the herein presented method gives rise to highly similar hBS-MP cell lines with a mesenchymal morphology and the potential to give rise to derivates of the paraxial mesoderm in vitro and in vivo. We present herein a universally applicable and reproducible method for deriving multiple hBS-MP cell lines from many different parental hBS cell lines and show for the first time that derivation and culture of hBS-MP can be performed under xeno-free conditions from a xeno-free parental hBS cell line (Ellerström et al., 2005). With respect to future clinical suitability we show that hBS-MP cells consistently survive transplantation and develop into several connective tissues in vivo, but do not give rise to teratoma.

Moreover, the cells presented herein are suitable also for in vitro applications, such as in different assays for detecting and/or measuring toxicity and or for use in the different stages of drug discovery and drug development. Today there are substances known to display inter-species differences and that can bring the effect of leading to severe malformations in humans like for example 13-cis retinoic acid (Isotretinoin) that is used in the treatment of acne (Accutane, Roche) and was not detected by toxicological tests based on mice (Anon et al, 1987; Hendrickx et al, 1998). One other substance with documented interspecies differences is thalidomide which caused severe malformations of new born children in the 1960s. Accordingly, human relevant developmental toxicity tests need to be established enabling the detection of so far unknown toxic substances as well as for obtaining more comprehensive data on substances with for instance known embryotoxic effects. Of great importance for enabling the development of such human relevant developmental toxicity tests, is the access to human relevant cell types that can be cultured under standard culture conditions in larger scale with reproducible results and low batch/lot variations and that can be plated and handled in multi-well culture plates for high throughput analysis. The hBS-MP cells presented herein provide these characteristics.

### SUMMARY OF THE INVENTION

One specific embodiment of the present invention relates to a novel mesenchymal human progenitor (hBS-MP) cell population derived from human blastocyst-derived stem (hBS) cells, said progenitor cell population having the following characteristics:
i) at least 80% of said cell population is negative for at least two markers reacting with undifferentiated hBS cells;
ii) at least 80% of said cell population is negative for at least one marker reacting with ectodermal lineage;
iii) at least 80% of said cell population is negative for at least one marker reacting with endodermal lineage;
iv) at least 30% of said cell population is positive for at least one marker reacting with mesodermal lineage and the marker being selected from vimentin and desmin.

In addition the herein presented invention relates to a method to obtain a human blastocyst-derived stem cell derived mesenchymal progenitor (hBS-MP) cell population, said method comprising;
i) plating of undifferentiated hBS cells onto a surface;
ii) incubation of the plated cells to allow differentiation;
iii) enzymatic passaging to a new surface;
iv) repeating of step (iii) until a homogenously mesenchymal morphology is obtained;
v) (optional) culture of obtained hBS-MP cells.

### DETAILED DESCRIPTION OF THE INVENTION

### Definitions and Abbreviations

“Assay” or “assays” are intended to describe in vitro tests performed to measure cytotoxicity and/or developmental toxicity on e.g. genetic, protein or functional level.

As used herein the term “marker” is used for markers for gene and/or protein expression, such as e.g. antibodies for use in for example immunocytochemistry, as disclosed in example 4 herein, or for example qrt-PCR, whatever must be relevant in the context.

As used herein “beta-tubulin”, “GFAP”, and “nestin” are intended to mean examples of markers known to react with the ectodermal cell lineage.

As used herein, the term “blastocyst-derived stem cell” is denoted BS cell, and the human form is termed “hBS cells”. In literature the cells are often referred to as embryonic stem cells, and more specifically human embryonic stem cells. The pluripotent stem cells used in the present invention can thus be embryonic stem cells prepared from blastocysts, as described in e.g. WO 03/055992 and WO 2007/042225, or be commercially available hBS cells or cell lines. However, it is further envisaged that any human pluripotent stem cell can be used in the present invention, including differentiated adult cells which are reprogrammed to pluripotent cells by e.g. the treating adult cells with certain transcription factors, such as OCT4, SOX2, NANOG, and LIN28 as disclosed in Junying Yu, et al., 2007.

As used herein, the term “de-differentiation” is intended to describe the potential of a cell or a cell type to convert into a less differentiated state.

As used herein the terms “hBS-MPs” or “hBS-MP cells” are intended to mean the mesenchymal progenitor cell population derived from hBS cells.

As used herein, the terms “E-cadherin” and “pan-cytokeratin” are intended to mean examples of markers reacting with epithelial cells.

By the terms “feeder cells” or “feeders” are intended to mean cells of one type that are co-cultured with cells of another type, to provide an environment in which the cells of the second type can grow. The feeder cells may optionally be from a different species as the cells they are supporting. The feeder cells may typically be inactivated when being co-cultured with other cells by irradiation or treatment with an anti-mitotic agent such as mitomycin c, to prevent them from outgrowing the cells they are supporting. Without limiting the foregoing, one specific such feeder cell type may be a human feeder, such as a human skin fibroblast, here denoted as hFF. Another feeder cell type may be mouse embryonic fibroblasts (mEF).

As used herein the terms “HNF3-beta” and “AFP” are intended to mean markers known to react with the endodermal cell lineage.

The term “IC50” value stands in the present context for the concentration of a test substance that leads to 50% death of tested cells in vitro.

The interpretation of the term “substance” is not intended to be limited to therapeutic agents (or potential therapeutic agents), or agents with documented toxicity effects such as neurotoxins, hepatic toxins, toxins of hematopoietic cells, myotoxins, carcinogens, teratogens, or toxins to one or more reproductive organs. The term substances may further be chemical compositions such as agricultural chemicals, e.g. pesticides, fungicides, fertilizers, or as well be components used in cosmetics.

As used herein the terms “progenitor” or “progenitor cell type” are any cell derived form hBS cells at any degree of differentiation between the undifferentiated hBS cell and a fully differentiated cell. More specifically, the progenitor cell referred to herein is a progenitor cell with mesenchymal feature and accordingly referred to as hBS-MP (hBS cell-derived mesenchymal progenitor) cell or hBS-MP cells in plural. Thus, in the present context, the term “progenitor stage” means the interval where the cells are in a proliferating phase as illustrated in FIG. 6, immature enough to be able to differentiate to several types of mesenchymal cell types.

“Efficiency” or “efficient”, if not otherwise defined, are in the context of an assay herein intended to mean that said assay is more likely to detect substances being toxic in human and/or that the toxic concentrations, such as the IC50 values analyzed, are closer to known human in vivo data than in methods or assays described in the prior art, such as assays based on mouse embryonic stem cells or mouse carcinoma cells.

As used herein “mesenchymal” is intended to mean belonging to the mesenchyme, the loose connective tissue of the developing organism which is of mesodermal origin.

As used herein the surface markers “CD105”, “CD166”, “CD10”, “CD13”, and “Stro-1” are intended to mean examples of markers for mesenchymal stem cells.

As used herein the surface markers “CD117” and “CD133” are intended to mean examples of markers for progenitor cells.

As used herein “SSEA-3, “SSEA-4, “Tra1-60”, “Tra1-80”, “Oct-4”, and “Nanog” are intended to mean examples of markers known to react with undifferentiated hBS cells.

As used herein, the terms “vimentin” and “desmin” and “ASMA” (or “alpha-SMA”) are intended to mean examples of markers known to react with the mesodermal lineage.

As used herein the term “xeno-free” is intended to mean never exposed to, directly or indirectly, material of non-human animal origin, such as cells, tissues, and/or body fluids and derivatives thereof.

As explained above, the present invention relates to human blastocyst-derived stem (hBS) cell-derived mesenchymal progenitor cells (hBS-MP cells) and one or more populations of such progenitor cells. The hBS-MP cell population has at least one, such as at least two, at least 3, at least 4 of the following characteristics:
i) at least 80%, such as at least 90% of said cell population is negative for at least two markers reacting with undifferentiated hBS cells;
ii) at least 80%, such as at least 90% of said cell population is negative for at least one marker reacting with the ectodermal lineage;
iii) at least 80%, such as at least 90% of said cell population is negative for at least one marker reacting with the endodermal lineage;
iv) at least 30%, such as at least 40% of said cell population is positive for at least one marker reacting with the mesodermal lineage and the marker being selected from vimentin and desmin.

By the term “at least X % of said cell population”, wherein X can be any percentage including 0 and 100, is referred to the percentage of a given cell type within a cell population consisting of two or more cell types. The cell population as described in the present invention consists of a close to 100% pure population, though it may under certain circumstances consist of a mixed population of cell types such as mesenchymal human progenitor cells, human blastocyst-derived stem cells or cell types from the other germ layers. For example, in the above the wording “A novel mesenchymal human progenitor (hBS-MP) cell population derived from human blastocyst-derived stem (hBS) cells, wherein: at least 80% of said cell population is negative for at least two markers reacting with undifferentiated hBS cells” is meant the percentage of mesenchymal human progenitor cells in the cell population consisting of a mixed population of cell types such as mesenchymal human progenitor cells and human blastocyst-derived stem cells. If so, in the given example at least 80% of the cells in the cell population are mesenchymal human progenitor cells.

The present invention further relates to an hBS-MP cell population, wherein at least 80% such as at least 90% of the hBS-MP cell population is negative for at least 3, such as at least 4, at least 5, at least 6 of the following markers reacting with undifferentiated hBS cells; SSEA-3, SSEA-4, Tra1-60, Tra1-80, Oct-4, and Nanog.

In further aspects, the present invention relates to an hBS-MP cell population, wherein at least 80% such as at least 90% or at least 95% of said cell population is negative for at least two, such as at least three, of the following markers reacting with the ectodermal lineage; beta-tubulin, GFAP, and nestin.

In further aspects, the present invention relates an hBS-MP cell population, wherein at least 80% such as at least 90% or at least 95% of said cell population is negative for at least one, such as at least two of the following markers reacting with the endodermal lineage; HNF3-beta and AFP.

In further aspects, the present invention relates to an hBS-MP cell population, wherein at least 50% such as, at least 60%, at least 70%, at least 80%, at least 90% or at least 95% of said cell population is positive for one or two of the following markers reacting with the mesodermal lineage; vimentin and desmin.

Although ASMA also is a mesodermal marker, the cell population according to the invention may in specific embodiments be an hBS-MP cell population, wherein less than 20%, such as less than 10% of said cell population is positive for the mesodermal marker ASMA.

In still further aspects, the present invention relates to an hBS-MP cell population, wherein less than 10%, such as less than 5% of said cell population is positive for the mesodermal marker ASMA.

As demonstrated in the examples herein, the cell population has a potential for increasing the percentage of positive reaction for the mesodermal ASMA marker.

In still further aspects, the present invention relates to an hBS-MP cell population, wherein at least 80%, such as at least 90% of said cell population is negative for at least one markers reacting with epithelial cells.

In still further aspects, the present invention relates to an hBS-MP cell population, wherein at least 80%, such as at least 90% of said cell population is negative for at least one of the following markers reacting with epithelial cells; E-cadherin and pan-cytokeratin.

In still further aspects, the present invention relates to an hBS-MP cell population, having the potential to give rise to a progeny cell population, wherein at least 80%, such as at least 90% of said progeny cell population is positive for at least two, such as at least three of the following markers reacting with the mesodermal lineage; vimentin, desmin, and ASMA.

Further analysis of the hBS-MP cell population by flow cytometry analysis, as described in Example 10 herein, for a set of accepted markers e.g. the progenitor cells (CD117, CD133) and the mesenchymal stem cells (CD105, CD166, CD10, CD13, Stro-1) markers, showed that a clear distinction from the population of undifferentiated hBS cell lines could be made.

Thus in a further aspect of the invention, the present invention relates to a hBS-MP cell population, wherein
i) at least 80% of said cell population express the mesenchymal stem cell markers CD166 and CD105,
ii) at least 60% of said cell population express the mesenchymal stem cell markers CD10, CD13 and Stro-1, and/or
iii) less than 10% of said cell population express the stem cell markers CD133 and CD117.

In a specific embodiment the present invention relates to an hBS-MP cell population, wherein at least 90% such as at least 95% or at least 99% of said cell population express the mesenchymal stem cell markers CD166 and CD105.

In another specific embodiment the present invention relates to an hBS-MP cell population, wherein at least 75% such as at least 80% or at least 90% of said cell population express the mesenchymal stem cell markers CD10, CD13 and Stro-1.

In a still further embodiment the present invention relates to an hBS-MP cell population, wherein less than 5% such as less than 2.5% of said cell population express the progenitor markers CD133 and CD117.

In still further aspects, the present invention relates to an hBS-MP cell population, wherein at least 50%, such as at least 60%, at least 70%, at least 80%, at least 90% of said cell population shows the following characteristics; a typical fibroblast-like morphology, i.e. elongated spindle-shaped cell morphology with branching pseudopodia (temporary projections) and an elliptic nucleus.

The present invention in additional aspects relates to an hBS-MP cell population further having the potential to form structures of one or more mesenchymal tissues and/or tissues derived from mesenchymal tissue in vitro and/or in vivo.

The ability of the hBS-MP cells to form tissues under three dimensional culture conditions in vitro may be tested by making aggregates of hBS-MP cells, which can be obtained by a short centrifugation step and subsequent culture in suspension culture for a suitable amount of days. The formed aggregates may then be examined macroscopically and further embedded in paraffin, cross-sectioned and stained for histology. Microscopic evaluation of obtained sections may then show homogenous tissue with spindle-shaped cells embedded in a large quantity of diffuse extracellular matrix (ECM) within the tissue sphere and a thin layer of elongated cells at the surface of the sphere. In general is expected to find that the tissue exhibit a high ECM to cell ratio, which is typical for the mesenchymal cell lineage.

The present invention in additional aspects relates to an hBS-MP cell population having the potential to form structures that resemble any of the following: connective tissue, cartilage, tendon or smooth muscle.

The present invention further relates to an hBS-MP cell population, wherein said cell population does not de-differentiate, a characteristic that for instance may be tested by transferring the hBS-MP cells back to a system for culturing undifferentiated hBS cells.

In addition the hBS-MP cell population according to the present invention may not give rise to hBS cell-typical teratoma formation, i.e. with the three germ layers present, when being engrafted into an immuno-deficient mouse, if for instance being engrafted under the kidney capsule of a SCID (severely combined immuno deficient) mouse. The teratoma tissue may instead be purely mesodermal.

The cell population according to the present invention may in one embodiment of the present invention be cultured without feeder cells and without conditioned medium present. In one specific embodiment of the present invention, the hBS-MP cell population is cultured directly on plastic, such as tissue culture treated plastic. One further aspect of the hBS-MP cell population is their ability to be passaged in split ratios of between 1:2 and 1:100, such as between 1:5 and 1:20, such as 1:10.

One embodiment thus relates to a method wherein the cell population can be passaged at a split ratio between 1:5 and 1:40, such as a split ratio of 1:10.

The present invention may in one embodiment relate to an hBS-MP cell population being more efficient in detecting or measuring the toxic effect of a substance than mature fibroblast cells. The hBS-MP cells may be from at least 1 to 5 times, such as at least 2 times more efficient in detecting or measuring the toxic effect of a substance than mature fibroblast.

Thus, in one embodiment the hBS-MP cell population may be used in detecting or measuring the toxic effect of a substance.

In one additional embodiment, the hBS-MP cells may be as efficient as undifferentiated hBS cells in detecting the toxic effect of a substance. In one specific embodiment of the present invention the hBS-MP cells may be as efficient as initially undifferentiated hBS cells in detecting the toxic effect of all-trans-retinoic acid.

In one embodiment the hBS-MP cell population may therefore be used in detecting or measuring the toxic effect of a substance when said substance is all-trans-retinoic-acid.

In one embodiment of the present invention, the hBS-MP cells may be xeno-free. Examples of procedures for establishment of a xeno-free hBS-MP cell population are presented below. Xeno-free hBS-MP cells, as well as the hBS cells used for obtaining the hBS-MP cells, may further be tested for Sialic acid Neu5Gc which is a membrane bound sugar molecule. A negative result in this test could be seen as an indication that no direct or indirect exposure to non-human animal material has occurred.

In still further embodiments, the cells may be genetically modified, with specific genes being either knocked-in or knocked-out. A marker gene under influence of a suitable promoter may then be transfected into either the hBS cells giving rise to the hBS-MP cells or into hBS-MP cells in the actual progenitor stage.

### Method

In further aspects of the present invention relates to a method to obtain a human blastocyst-derived stem cell derived mesenchymal progenitor (hBS-MP) cell population. The method may comprise at least 2, such as at least 3, at least 4, such as 5 steps. In one specific embodiment of the present invention, the method to obtain an hBS-MP cell population comprises the steps of:
i) plating of undifferentiated hBS cells onto a surface;
ii) incubation for between 2 and 21 days, such as for 3 to 10 days, to allow differentiation;
iii) enzymatic passaging to a new surface;
iv) repeating of step (iii) until a homogenously mesenchymal morphology is obtained;
v) (optional) culture of obtained hBS-MP cells.

In one embodiment according to the invention the surface in step i) and/or step ii) is a tissue culture treated plastic or is a surface coated with a substance selected from mixed ECM extracts such as gelatin, Matrigel™, human placental matrix, or purified/synthetic ECM compounds, such as collagen, heparin sulfate, laminin, fibronectin, or combinations thereof.

By the term “enzymatic passaging” means passaging of cells by enzymatic treatment, such as with trypsin, TrypLE™, select, accutase alone or in combination with Ca-chelator eg. EDTA, where the cells are dissociated to single cells as well as clusters of varying size.

Accordingly, in one embodiment the present invention relates to method wherein the enzymes used in step iii) are selected from the group consisting of trypsin, TrypLE™, select, accutase alone or in combination with Ca-chelator, such as eg. EDTA.

The incubation in step (ii) may take place until outgrowths of heterogeneous cell types occur in the plated cell cultures.

A homogeneous mesenchymal morphology in step (iv) is intended to mean that a majority of the cells have fibroblast like, i.e. elongated spindle-shaped cell morphology with branching pseudopodia (temporary projections) and an elliptic nucleus.

In one specific embodiment of the present invention, the method to obtain a human blastocyst-derived stem cell derived mesenchymal progenitor (hBS-MP) cell population comprises;
i) plating of undifferentiated hBS cells onto a gelatin coated surface;
ii) incubation of the plated cells for 5 to 7 days, until outgrowths of heterogeneous cell types occur;
iii) enzymatic single cell suspension passaging to a new gelatin coated surface;
iv) repeating of step (iii) until a homogenously mesenchymal morphology is obtained;
v) (optional) further culture of obtained hBS-MP cell population.

In one embodiment according to the invention the plated cells in step ii) are incubated for at least 5 days, such as e.g. 5-9 days or e.g. 7 days to allow differentiation until outgrowths of heterogeneous cell types occur.

In another embodiment according to the invention the plated cells in step ii) are incubated for 5 to 7 days, such as e.g. 7 days until outgrowths of heterogeneous cell types occur.

One additional feature of the herein presented invention is that no selection of cells is necessarily done in steps (iii) and (iv). The herein presented method provides a significantly facilitated approach compared to previous presented methods in obtaining homogenous mesenchymal cells from differentiating hBS cells, since the herein claimed method eliminates the need of co-culturing, cell sorting, manual selection of specific cell types at passage as well as transfections.

Furthermore, the herein presented method can be performed by passaging of single cells. Passaging and seeding of single cells increases the reproducibility of the method and allows low spreads between individual culture vessels, since the cells can actually be counted and more exact numbers seeded and/or used.

Thus, in one embodiment the present invention also relates a method, wherein the cells after enzymatic treatment in the enzymatic passaging step ii) are in the form of a single cell suspension, as discussed further below.

The term “enzymatic single cell passaging” used herein means enzymatic cell passage with an enzyme such as, trypsin, TrypLE™, select, accutase alone or in combination with Ca-chelator eg. EDTA, where the cells are dissociated to mostly single cells, though the suspension may contain small clusters of up to 20 cells, such e.g. 10, 8 or 4 cells.

As starting material to obtain hBS-MP cells can be used traditionally culture hBS cells on mEF (Heins. et. Al; 2004) or enzymatically culture hBS cells growing on a protein coating or on other feeder cell types, such as human feeders. The cells may be detached mechanically or detached and dissociated enzymatically prior to the plating in step (i) above. In a specific embodiment of the present invention, hBS cells were detached mechanically from mEF by use of a sharp microcapillary and transferred to gelatin coated tissue culture plates.

In one embodiment of the present invention the culture medium used in the herein presented method is chosen from a group comprising, but not limited to, Vitrohes™, Vitrohes™ with bFGF and hBS-MP cell medium. hBS-MP medium typically contains a base medium, such as DMEM (Dulbecco's Modifeid Eagle's Medium), supplemented with between 1% (v/v) and 20% (v/v) FBS, and with between 1 ng/ml and 100 ng/ml bFGF. In one specific embodiment of the present invention the hBS-MP medium contains DMEM with high glucose and without pyruvate, 10% (v/v) FBS, 10 ng/ml bFGF (all from Invitrogen).

One embodiment of the invention therefore relates to a method wherein the culture medium used is chosen from a group which supports the differentiation towards and/or proliferation of mesenchymal progenitor cells selected from a group comprising, but not limited to, Vitrohes™, Vitrohes™ with bFGF, human recombinant FGF, FBS and hBS-MP cell medium, a mammalian cell culture medium such as IMDM, DMEM, DMEM/F12 in combination with serum, such as fetal bovine serum or human serum.

By the above term “hBS-MP medium” is meant a medium consisting of a mammalian cell culture medium such as IMDM, DMEM, DMEM/F12 in combination with bFGF or human recombinant FGF in the rage of 0, 1-100 ng/ml, such as 2-0 ng/ml, such as e.g. 4-10 ng/ml and FBS in the range 1-40%, such as e.g. 10-20%.

Another embodiment of the invention thus relates to a method wherein the concentration of bFGF or human recombinant FGF is in the rage of 0.1-100 ng/ml, such as 2-0 ng/ml, such as e.g. 4-10 ng/ml.

A further embodiment of the invention thus relates to a method wherein the concentration of FBS is in the range 1-40%, such as e.g. 10-20%.

Using the above described method of consecutive enzymatic passaging as single cell suspensions under the described conditions reproducibly led to the derivation of morphologically homogeneous hBS-MP cell lines from cultures of pluripotent undifferentiated hBS cells within 2-3 passages (see FIG. 1). The plated hBS cells initially gave rise to a mixed population of various differentiating cell types (see FIG. 1a). Each consecutive passage decreased the amount of contaminating cell types and the cultures became increasingly homogeneous (FIGS. 1B, C). The hBS-MP cells phenotypically resemble mesenchymal cells, i.e. having an elongated spindle-shaped cell morphology with branching pseudopodia (or temporary projections) and an elliptic nucleus.

The method developed derives hBS-MP cell lines from undifferentiated hBS cell lines due to a selection pressure which favors fast growing cells with an ability to attach and proliferate in feeder-free monolayer culture. The consecutive passaging provide a growth advantage for hBS-MP cells while slow-growing differentiated cell types as well as undifferentiated hBS cells are eliminated. The protocol eliminates the need for embryoid body formation, cell transfection, co-culture, cell sorting or subjective manual selection of certain cell types, the latter normally involving for instance a step of visual inspection and directed mechanical and/or enzymatic detachment of desired cell types to derive hBS-MP cells.

Thus in one embodiment of the present invention, a method is provided wherein consecutive passaging and the incubation time in steps ii) and iii) leads to conditions which allow the selective survival and proliferation of hBS-MPs to maintain already formed hBS-MP cells to proliferate, without significant differentiation.

In another embodiment of the present invention, a method is provided wherein the selection pressure applied avoids additional selection of hBS-MP cells in step iii) and/or step iv).

By the above term “additional selection” is meant manual selection or selection by protein markers, such as antibodies, FACS, magnetic beads, selection by genetic markers, such as transfection, antibiotic selection. The selection in the invention is defined, as selection and maintenance of pure hBS-MPs populations by the culture method requiring no additional selection.

In still another embodiment of the present invention, the hBS-MP cells are derived and cultured according to a xeno-free protocol with only xeno-free, synthetic or human recombinant components being used in the different steps. For xeno-free derivation of an hBS-MP cell population, the hBS cell line from which the hBS-MP cells are to be derived also needs to be xeno-free. In addition, the individual components of the system, need to be exchanged to components being either synthetic, human recombinant or otherwise xeno-free, exemplified by, but not limited to, exchanging FBS to human serum, and exchanging bovine gelatin to recombinant gelatin for protein coating of culture vessels. Xeno-free derivation and culture of a xeno-free hBS-MP cell population may also be performed by applying a completely defined culture medium with recombinant or synthetic culture components added in known amounts.

Thus, embodiment according to the present invention relates to a method wherein all reagents, such as e.g. media, growth factors, feeder cells, and other materials used are xeno-free in order to obtain xeno-free hBS-MP cells.

In still further aspects, the hBS-MP cells as well as the xeno-free hBS cells may according to the present invention be used for GMP production, such as clinical GMP production of hBS-MP cells and/or differentiated cells thereof. The herein described method for xeno-free derivation and culture of hBS cells and hBS-MP cells is then performed under GMP and/or cGMP conditions to provide clinically applicable cell lines and derivatives.

Furthermore, the present invention relates to a method of further differentiating the hBS-MP cells to for instance more mature mesodermal cell types. One suitable method for such differentiation is presented in the examples below, but a general approach can be to leave the cells in the same culture vessel for a prolonged time without passage and optionally switch to another medium formula. One additional such approach can be to let the cells differentiate in their “old” medium by performing less frequent medium changes to the cultures.

The production and manipulation of hBS cells may be scaled up using novel culture systems for bulk culture. The presented derivation and cultivation methods for hBS-MP cells do not require any manual selection, micromanipulation, or co-culture and can therefore be automated. Suitable robots could be based on XYZ dispensing heads such as used in liquid handling stations which allow pipetting to and from culture vessels. Automation could alternatively be based on a robotic arm which mimics the movements of a human being during culture vessel and pipette handling.

Further scale up of hBS-MP derivation and cultures can be achieved by the use of bioreactors, such as hollow fibre reactors, perfused reactors or stirred reactors. Such bioreactors should maintain environmental parameters such as e.g. temperature, nutrient supply, pH, pressure, shear forces, oxygen within optimal limits and can be driven in batch, fed batch or continuous operation. The hBS-MP cells may then be grown on for instance hollow fibre capillary membranes or as attached to microcarriers. This system would enable growth of the cells into larger cell masses between the capillaries, and provide an optimized, natural environment by the perfusion of culture medium and gases like oxygen. The closed bioreactor systems may be developed to produce larger amount of cells, and to support the potential maintenance of functional properties of the cells. Cell isolation via enzyme perfusion would then allow scale-up in closed GMP systems. Cell purification could then be performed using e.g. FAC sorting based on e.g. membrane antigen expression or using density gradient media and centrifugation.

### Use

In still further aspects the present invention relates to the use of the hBS-MP cell population as defined herein for use in the drug discovery process, such as for studying drugs with a potential effect on mesenchymal cell types.

The hBS-MP cells of the present invention may as well be used for studying genesis of mesenchymal tissues, such as, e.g., early cartilage or connective tissue.

The hBS-MP cells of the present invention may in still further aspects be used for studying human degenerative disorders.

In still further aspects, the hBS-MP cells of the present invention may be used for in vitro toxicity testing. Assays in which the cells may be used are exemplified by, but not limited to, assays for in vitro toxicity for the detection and/or prediction of toxicity in the human species, wherein the individual assay enables novel detection of toxicity for a substance and/or more efficiently detects toxicity compared to non-human assays or assays based on adult human cell types. Suitable endpoints in such a toxicity assay may be embryo toxic and examples of suitable embryo toxicity endpoints are measurements of gene and protein expression. In addition, the endpoints chosen may be cytotoxic detecting and/or measuring cell viability vs. cell death. One way to visualize cytotoxicity is to measure the metabolic activity of cells, exemplified by but not limited to resazurin conversion, MTT salt analysis and ATP content analysis.

To perform the assay, the cells may be dissociated into small cell aggregates, or preferably, single cells and seeded into multi-well-format plates, such as 96-well plates in suitable volumes of a test medium. After a couple of hours or days, the cytotoxicity test may be started by adding the toxicity solution to the test wells. Toxicity medium may thereafter be changed frequently throughout the assay and the plates finally analyzed by measuring different endpoints for e.g. cytotoxicity, such as metabolic assays for ATP content, Resazurin conversion and/or MTT salt content. If then comparing obtained IC50 values for certain substances on different cell types, such as hBS cells undifferentiated from start, hBS-MP cells and for instance a fibroblast cell type may then show which cell type is more sensitive or efficient in detecting or measuring a toxic effect of the substance. The hBS cells and hBS-MP cells may be more sensitive towards certain substances than for instance fibroblasts. The progenitor cells hence may represent one more easily cultured hBS cell type enabling larger scale culture with enzymatic passaging while maintaining a higher sensitivity to toxic substances.

In a further embodiment, the present invention relates to use of the hBS-MP cell population in regenerative medicine and/or in medicine, such as for the manufacture of a medicinal product for the prevention and/or treatment of pathologies and/or diseases caused by tissue degeneration, such as, e.g., the degeneration of mesenchymal tissue. In one further aspect, the present invention relates to the use of the hBS-MP cells for the manufacture of a medicinal product for the prevention and/or treatment of connective tissue disorders.

In still further aspects, the present invention relates to use of the hBS-MP cells for obtaining mesodermal cell types from a group comprising, but not limited to, chondrocytes, myocytes, and osteocytes and/or for studying maturation towards mesodermal cell types, such as studying maturation towards connective tissue cells.

The hBS-MP cells, no longer being undifferentiated hBS cells as shown by marker analysis and, accordingly, not giving rise to tumours in vivo, still being able to proliferate and still having a potential to differentiate into several mesenchymal cell types (including chondrocytes and connective tissue cells), may be suitable for treating the majority of disorders and diseases by reversing, inhibiting or preventing tissue damage.

The hBS-MP cells may be used for treating disorders associated with, for example, necrotic, apoptotic, damaged, dysfunctional or morphologically abnormal connective tissue.

In addition the cells may be used for reconstructive medicine and cell replacement therapy concerning fat, bone, muscle, tendon, and cartilage or for degenerative diseases, acute injuries and plastic surgery.

The hBS-MP cells may for instance be used for restoring joint function and replacing articular cartilage, which has a limited potential to repair. Unsatisfactory results with current treatment methods (e.g. osteochondral autografts, drilling or microfracturing) has triggered the development of new cartilage restoration techniques including autologous cell transplantation with mesenchymal stem cells or chondrocytes. The hBS-MP cells can in this context provide one such additional therapy by providing an homogeneous material from an unlimited and defined source. Xeno-free hBS-MP cells may be derived under GMP conditions and according to xeno-free protocols as presented herein and further expanded and subject to differentiation towards the desired cell type. The administration of the cells may then be performed with or without a supporting scaffold. The mesodermal origin of the cells further makes them possible for deriving other mesodermal tissue, such as blood, blood vessels, and cardiac cells.

In still further aspects the hBS-MP cells can be transplanted to humans as a treatment or in the frame of plastic or reconstructive surgery, preferably comprises treating the subject with an immunosuppressive regimen, preferably prior to such administration, so as to inhibit such rejection.

The cells may further be used for GMP production of xeno-free human extracellular matrix components and human biologicals in vitro, such as e.g. cytokines which promote tissue repair, growth factors, vaccines, viruses and proteins.

The hBS-MP cells or cells derived from thereof may further be used for treating disorders associated with, for example, necrotic, apoptotic, damaged, dysfunctional or morphologically abnormal myocardium. Such disorders include, but are not limited to, ischemic heart disease, cardiac infarction, rheumatic heart disease, endocarditis, autoimmune cardiac disease, valvular heart disease, congenital heart disorders, cardiac rhythm disorders, and cardiac insufficiency.

The cells, no longer being undifferentiated hBS cells as shown by marker analysis and, accordingly, not giving rise to tumour formations in vivo, being able to proliferate and having a potential to differentiate into also cardiac cell types (including cardiomyocytes, endothelial cells and smooth muscle cells), may therefore be suitable for treating the majority of cardiac disorders and diseases by reversing, inhibiting or preventing cardiac damage caused by ischemia resulting from myocardial infarction.

In still further aspects the hBS-MP cells and cells derived thereof can be used to treat cardiac disorders characterized by abnormal cardiac rhythm, such as, for example, cardiac arrhythmia. The treatment is preferably performed by administering a therapeutically effective dose of the cells to the heart of the subject, preferably by injection into the heart. A therapeutically effective dose is an amount sufficient to generate a beneficial or desired clinical result, which dose could be administered in one or more administrations. The injection can be administered into various regions of the heart, depending on the type of cardiac tissue repair required. The administration may be performed using a catheter-based approach after opening up the chest cavity or entry through any suitable blood vessel. The effective dose of cells can be based on factors such as weight, age, physiological status, medical history, infarct size and elapsed time following onset of ischemia. The administration of hBS-MP cells preferably comprises treating the subject with an immunosuppressive regimen, preferably prior to such administration, so as to inhibit such rejection.

In still further aspects the hBS-MP cells may be used for conditioning of culture medium for e.g. culture of undifferentiated hBS cells or as feeder cells for such cultures, as described in example 12. This gives the advantage of having feeders that potentially could be derived from the same hBS cell line as the undifferentiated hBS cells cultured, which lowers the contamination risk etc. Specifically transfected hBS-MPs can be of certain advantage for use as tagged feeder cells, example 13.

The hBS-MP cells may for instance be used for restoring joint function and replacing articular cartilage, which has a limited potential to repair. Unsatisfactory results with current treatment methods (e.g. osteochondral autografts, drilling or microfracturing) has triggered the development of new cartilage restoration techniques including autologous cell transplantation with mesenchymal stem cells or chondrocytes. The hBS-MP cells can in this context provide one such additional therapy from a defined source and with reproducible characteristics.

Finally, the present invention relates to a kit for deriving and/or culturing hBS-MP cells, said kit comprising:
i) undifferentiated hBS cells;
ii) one or more culture media, chosen from a group comprising, but not limited to, Vitrohes™, Vitrohes™ with bFGF, hBS-MP cell medium;
iii) one or more suitable enzymes, and
iii) optionally, an instruction for use.

One additional kit type of the present invention relates to a kit for regenerative medicine comprising:
i) hBS-MP cells;
ii) optionally, factors for driving differentiation in vitro and/or in vivo;
iii) tool(s) for administration of the cells to a patient or cells in an administrative form, such as in a ready-to-use syringe.

A third example of a kit according to the present invention is a progenitor-cell based kit for detecting toxicity in human, said kit comprising:
i) hBS-MP cells;
ii) (optional) positive and negative control substances;
iii) one or more reagents for detecting and/or measuring cytotoxicity;
iv) (optional) an instruction for use.

### REFERENCES

Thomson J A, Itskovitz-Eldor J, Shapiro S S et. al. (1998) Embryonic stem cell lines derived from human blastocysts. Science 1998; 282:1145-1147
Sottile F, Olevano V, Reining L. (2003) Parameter-free calculation of response functions in time-dependent density-functional theory. Phys Rev Lett. 2003 Aug. 1; 91(5):056402. Epub 2003 Jul. 31.
Xu Chunhui, Jaing Jianjie, Sottile Virginie, Mc Whir Jim, Lebkowski Jane, Carpenter Melissa K., (2004), Immortalized Fibroblast-Like Cells Derived from Human Embryonic Stem Cells Support Undifferentiated Cell Growth, Stem Cells 2004; 22:972-980
Lian Qizhou, Lye Elias, Yeo Keng Susan, Khia Way Tan Eileen, Salto-Tellez Manuel, Lui Tong Ming, Palanisamy Nallasivam, Menshawe El Oakley Reida, Lee Eng Hin, Lim Bing, Lim Sai-Kiang, Derivation of Clinically Compliant MSCs from CD105+, CD24− Differentiated Human ESCs, Stem Cells 2006 published online Oct. 19, 2006
Stojkovic Petra, Lako Majlinda, Stewart Rebecca, Przyborski Stefan, Armstrong Lyle, Evans Jerome, Murdoch Alison, Strachan Tom, Stojkovic Miodrag, (2005), An Autogenic Feeder Cell System That Efficiently Supports Growth of Undifferentiated Human Embryonic Stem Cells, Stem Cells 2005; 23:306-314
Oliver Emmanuel, Rybicki Anne C., Bouhassira Eric E., (2006) Differentiation of Human Embryonic Stem Cells into Bipotent Mesenchymal Stem Cells, Stem Cells 2006; 24:1914-1922
Ellerström Catharina, Strehl Raimund, Moya Karina, Andersson Katarina, Bergh Christina, Lundin Kersti, Hyllner Johan, Semb Henrik, Derivation of a xeno-free human ES cell line, Stem cells express 2006; doi: 10.1634/stemcells. 2006-0130
Anon et al, Teratology Society position paper: recommendation for vitamin A use during pregnancy. Teratology, 1987 2, 269-275
Hendrickx et al, Teratogenecity of retinoids in rodents, primates, and humans. J. Toxicol Sci 4, 1998, 272-272)
Heins N, Englund M C O, Sjöblom C et al., Derivation, characterization, and differentiation of human embryonic stem cells. Stem Cells 2004; 22:367-376.
Heins N, Lindahl A, Karlsson U, Rehnström M, Caisander G, Emanuelsson K, Hanson C, Semb H, Björquist P, Sartipy P, Hyllner J., Clonal derivation and characterization of human embryonic stem cell lines., Journal of Biotechnology 2005 Nov. 29.
Gertow K, Wolbank S, Rozell B, Sugars R, Andäng M, Parish C L, Imreh M P, Wendel M, Ahrlund-Richter L., Organized development from human embryonic stem cells after injection into immunodeficient mice., Stem Cells Dev. 2004 August; 13(4):421-35.
WO2003055992: A method for the establishment of a pluripotent blastocyst-derived stem cell line, Cellartis AB
WO2005059116: Methods for clonal derivation of human blastocyst-derived stem cell lines, Cellartis AB

### FIGURE LEGENDS

FIG. 1 shows fast growing progenitor cells developing as one cell type in a heterogeneous culture. After subsequent passage the fast growing cells become dominant, i.e. hBS-MP cells are formed (A-C) Establishment and culture of hBS-MPs from hBS cell line SA002.5. (A) in passage 0 seeded in high density (150.000 cell/cm2), (B in passage 2, and (C) in passage 7. (See example 1 and 2.)

FIG. 2 shows xeno-free progenitors at first passages after establishment from hBS cell line SA611 (A) in passage 2 (B) in passage 4. (See example 3.)

FIG. 3 shows the hBS-MP cells stained with an alpha-human nuclei antibody proving the human origin of the cells. (See example 2.)

FIG. 4 shows immunocytochemical markers expressed in hBS-MP cell cultures in continuous culturing (A-C) and after differentiation (D-F). (See example 4 and 5.) (A), and (D) show desmin, (B) and (E) show vimentin, (C) and (F) show ASMA.

FIG. 5 shows in vivo differentiation of hBS-MP cells with only mesenchymal derivatives present. The size of the developed tissue is a lot smaller compared to the size of hBS cell teratomas, indicating limited growth of the hBS-MP cells after transplantation. (A) cartilage, (B) confirmation of cartilage by alcain blue/van Giessen, (C) confirmation of cartilage by safranin orange staining (D) mixed mesodermal structures, cartilage, immature smooth muscle, loose connective tissue, (E) immature tendon and (F) for comparison, teratoma developed from undifferentiated hBS cells with the three germ layers represented. (See example 7.)

FIG. 6 shows a schematic figure of the progenitor stage; whilst maturing cells can be said to pass the progenitor stage, a stage occurring for a longer or shorter time depending on cell type. When cultured as described here, the hBS-MP cells stay in the progenitor stage, as a cell type still with potential to mature into more specific tissues in vivo, though with lost capability to form structures from all germ layers, referred herein to as teratomas.

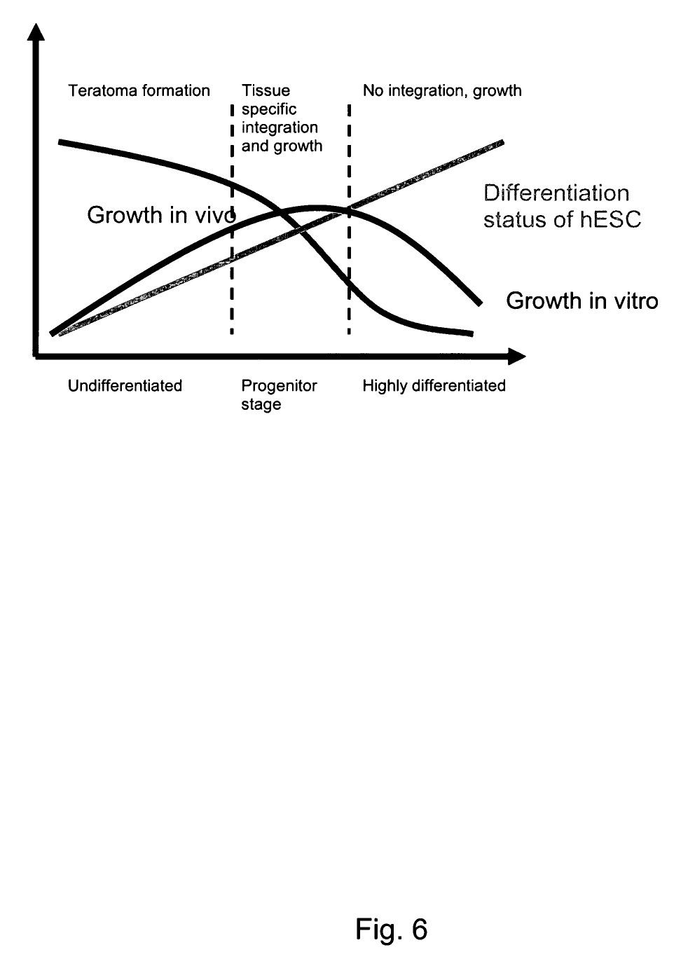

FIG. 7 (A) shows the dose-response curve for 13-cis-retinoic-acid (13CRA) in hBS cells, hBS-MP cells and hFF. The hBS cells and the progenitor cells show a higher sensitivity to the toxic substance than the hFF. The IC50 ratios between hBS and progenitor cells and hBS cells and hFF are 1:4 and 1:10, respectively. The data were obtained by measuring the ATP content per well and was normalized to the untreated/solvent control. (B) shows the dose-response curve for all-trans-retinoic-acid (ATRA) in hBS cells, hBS-MP cells and hFF. The hBS cells and the progenitor cells show a higher sensitivity to the toxic substance than the hFF. The data were obtained by measuring the ATP content per well and was normalized to the untreated/solvent control. (See example 9.)

FIG. 8 Representative flow cytometric analysis of hBS-MP cells and undifferentiated hES cells from the same cell line (SA002.5) with regard to markers for stem cells (CD117, CD133) and mesenchymal stem cells (CD105, CD166, CD10, CD13, Stro-1). Grey histograms; hES-MP cells, white histograms; undifferentiated hES cells, line; fluorescence intensity considered as positive signal obtained from isotype controls).

FIGS. 9 (A-C) Shows hBS-MPs as feeder cells. hBS cell-line SA002.5 cultured on hBS-MPs derived from hBS cell-line SA002 in passage 5. The cells were enzymatically dissociated to single cells for 10 consecutive passages.

FIGS. 10 (A-C) shows osteogenic differentiation of hBS-MPs. Mallory Aniline Blue staining of hBS-MP cell line 2.5 cultured in β-tricalcium phosphate ceramic for 6 weeks. The staining showed large orange to red areas (indicated with arrows) corresponding to mineralized bone tissue, see example 11 Osteogenic differentiation model.

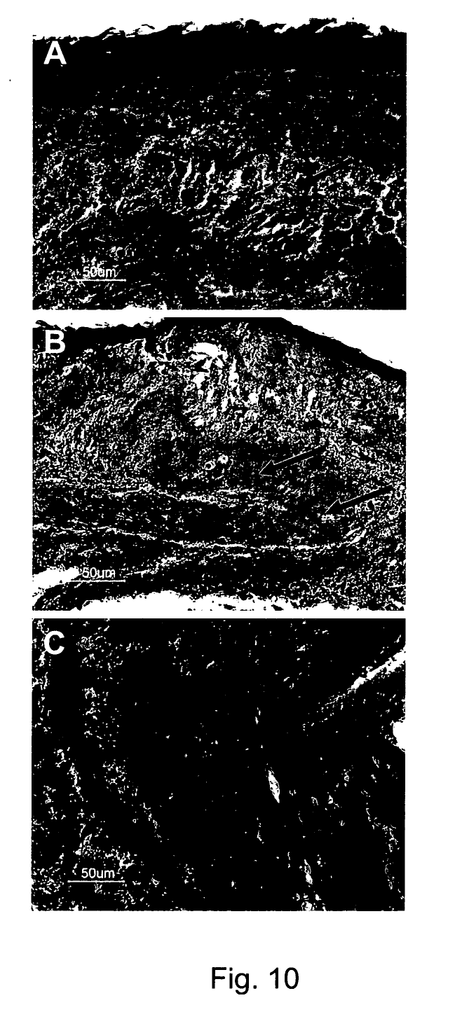

FIG. 11 shows transfected hBS-MPs with stable integrated clones expressing red fluorescent protein, see example 13, Transfection of hBS-MPs.

### EXAMPLES

### Example 1

### Starting Material

The starting material for the present invention is suitably pluripotent undifferentiated hBS cells, such as undifferentiated hBS cell lines. Such material can be obtained from Cellartis AB and is also available through the NIH stem cell registry http://stemcells.nih.gov/research/registry/. Cellartis AB has two hBS cell lines (SA001 and SA002) and one subclone of SA002 (SA002.5) available through the NIH. All the hBS cell lines used are approved and registered by the UK Stem Cell Bank Steering Committee and SA001, SA002, SA002.5 and SA611 are as well approved by MEXT (Japan).

Those hBS cell lines have been frequently used in the present invention. Characteristics of the hBS cells recommended as starting material are the following: positive for alkaline phosphatase, SSEA-3, SSEA-4, TRA 1-60, TRA 1-81, Oct-4, negative for SSEA-1, telomerase activity, and pluripotency in vitro and in vivo (the latter shown by teratoma formation in immuno-deficient mice). (Methods and protocols as previously shown, Heins et al, WO2003055992.)

For the derivation of hBS-MP cell lines nine different hBS cell lines were used. All hBS cell lines had been established and characterized at Cellartis AB, Gothenburg Sweden. The establishment, clonal derivation, characterization and subsequent culture of hBS cell lines SA001, SA002, SA002.5, AS034, SA121, SA167, SA348 and SA461 had been carried out as described previously (Heins et al., 2004; Heins et al., 2005; WO03055992, WO2005059116). The xeno-free hBS cell line SA611 had been established, expanded and characterized as recently described (Ellerström et. al. 2006).

### Example 2

### Derivation of hBS-MP Cell Lines and Subsequent Culture of the hBS-MP Cell Lines

Undifferentiated hBS cells were removed from the supporting feeder layer, enzymatically dissociated and plated onto 0.1% porcine gelatin coated cell culture dishes (BD Biosciences, Bedford, Mass., USA) at 1.5×10⁵ cells per cm² in hBS-MP medium consisting of DMEM (high glucose with glutamax, without pyruvate)+10% fetal bovine serum (FBS)+10 ng/ml human recombinant basic fibroblast growth factor (hrbFGF) (all from Gibco/Invitrogen). The plated hBS cells were left to differentiate, i.e. no medium changes or passaging of cells to fresh culture environments, for 7 days in a humidified atmosphere at 37° C. and 5% CO₂ resulting in a outgrowth of heterogeneous cell types. To initiate the derivation of hBS-MP cells the hBS cells were then passaged enzymatically (passage 1, p1) as a single cell suspension using TrypLE Select (Gibco, Invitrogen) to new gelatin coated culture dishes. In this and in all following passage or transfer steps no selection was performed manually, i.e. all cells were transferred. This procedure was repeated every 7 days until the cell population became homogeneous for hBS-MP morphology (at p2-p3). Following the initial derivation steps the hBS-MPs were cultured in tissue culture flasks (Falcon, BD Biosciences) in a humidified atmosphere at 37° C. and 5% CO₂ and enzymatically passaged with TrypLE Select (Gibco, Invitrogen) every 7 days at a split ratio of 1:10.

Regarding the derivation of hBS-MPs have successfully been carried out on tissue culture treated plastic polystyrene plates as well as on laminin coated plates, data not shown.

The advanced method we developed to derive hBS-MP cell lines from undifferentiated hBS cell lines is based on the application of a selective pressure which favors fast growing cells with an ability to attach and proliferate in feeder-free monolayer culture. We have found these conditions to provide a growth advantage for hBS-MP cells while slow-growing differentiated cell types as well as undifferentiated hBS cells are eliminated. The protocol eliminates the need for embryoid body formation, cell transfection, co-culture, cell sorting or subjective manual selection of certain cell types, the latter normally involving for instance a step of visual inspection and directed mechanical and/or enzymatic detachment of desired cell types to derive hBS-MP cells.

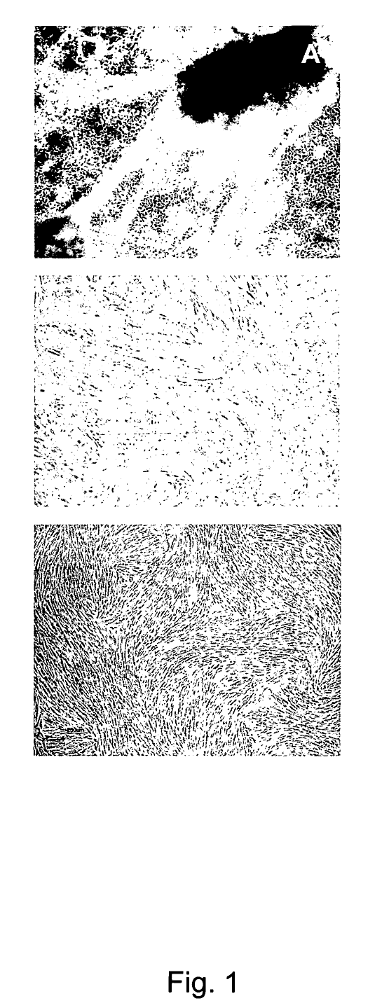

Consecutive enzymatic passaging as single cell suspensions under the described conditions reproducibly led to the derivation of morphologically homogeneous hBS-MP cell lines from cultures of pluripotent undifferentiated hBS cells within 2-3 passages (FIG. 1). The plated hBS cells initially gave rise to a mixed population of various differentiating cell types (FIG. 1a). Each consecutive passage decreased the amount of contaminating cell types and the cultures became increasingly homogeneous (FIGS. 1B, C). The hBS-MP cells phenotypically resemble mesenchymal cells, i.e. having an elongated spindle-shaped cell morphology with branching pseudopodia (or temporary projections) and an elliptic nucleus. To allow bulk production the hBS-MP cells were cultured in flasks and were passaged every 7 days at a split ratio of 1:10. At high densities when the cultures were ready for passage, the hBS-MP cells showed a more elongated morphology (FIG. 1D). Using this 7 day passage interval, the hBS-MP cell lines could be expanded massively for 16-20 passages before a decrease in proliferative capacity became apparent.

The protocols for the derivation of hBS-MP cell lines showed excellent reproducibility and robustness. Multiple hBS-MP cell lines have been successfully derived and cultured from eight different hBS cell lines, SA001, SA002, SA002.5, AS034, SA121, SA167, SA461 and xeno-free SA611.

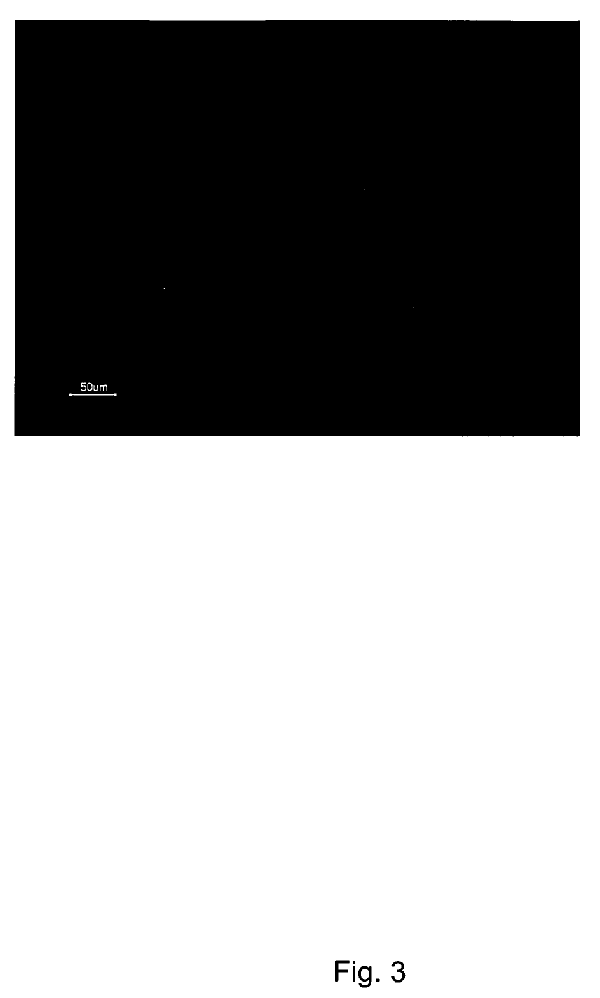

Due to the morphology and the fast expansion of the hBS-MP cells we initially considered the remote possibility that the observed fast growing cell populations may actually be mouse embryonic fibroblasts that had not been growth-inactivated sufficiently and had been transferred from the feeder layer by accident. To rule out that possibility we performed immunostainings with an antibody specific for human nuclei on hBS-MP cell cultures. These stainings clearly confirmed that all cells were human (FIG. 3, Table 1).

### Example 3

### Xenofree Modification for Derivation and Expansion of hBS-MP Cell Lines

In the xeno-free variant of the protocols FBS was replaced with human serum (HS) in the hBS-MP medium. Human serum was prepared and tested as described earlier (Ellerström et. al. 2006). Human recombinant gelatin (Fibrogen, San Francisco, Calif.) was used to coat culture dishes instead of porcine gelatin. Alternatively non-coated tissue culture flasks were used for expansion with similar efficiency.

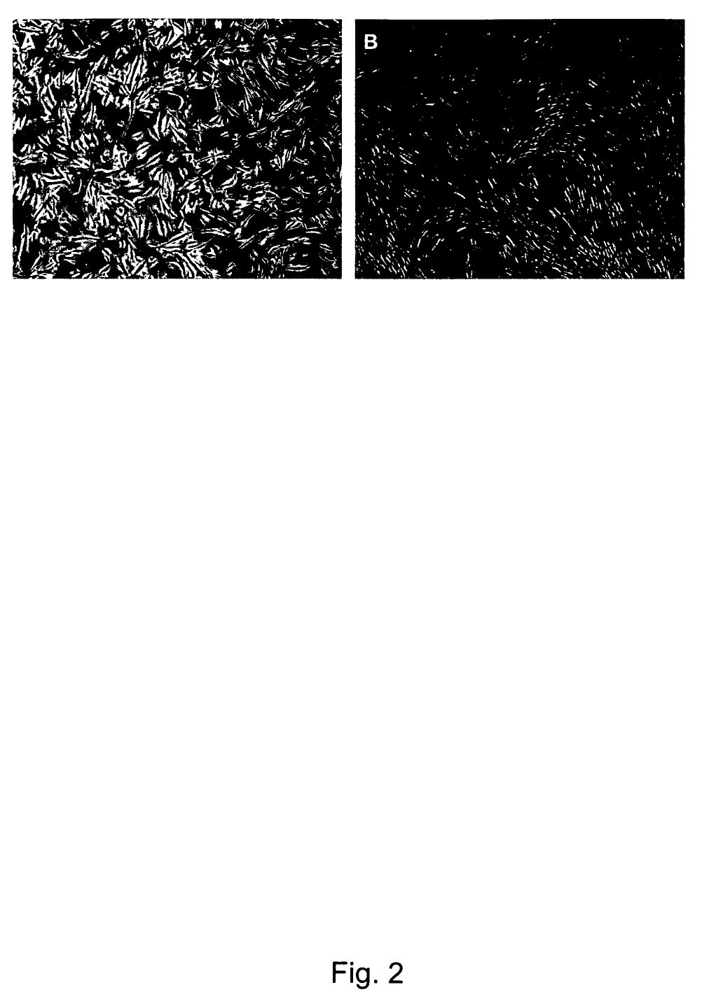

To derive hBS-MP cells from a xeno-free hBS cell line without contaminating the cell cultures with animal-protein all animal derived components were replaced with human derived or recombinant components. Human serum was used in the culture medium instead of FBS and human recombinant gelatin was used instead of porcine gelatin to coat culture dishes. Using these modified protocols a xeno-free hBS-MP cell line was derived from the xeno-free hBS cell line SA611 (Ellerström et al., 2006). Upon removal from the supporting human foreskin fibroblast (hFF) feeder layer and transfer to cultures in high density (around 150.00 cells seeded per cm²), the xeno-free hBS cells initially showed an outgrowth pattern with less distinct three dimensional structures. But already after two passages the arising xeno-free hBS-MP cells were morphologically indistinguishable from the previously established non xeno-free lines (FIG. 2).

### Example 4

### In Vitro Characterization by Immunocytochemistry

Cells were fixed in 4% paraformaldehyde solution for 10 minutes at room temperature, permeabilized with 0.5% Triton-X 100 Non-specific binding was blocked with 10% FBS in PBS (Gibco/Invitrogen) for 30 minutes before the primary antibodies, table 1a, were incubated for 20 h at +4° C. Negative controls were included in which the primary antibodies were omitted. The secondary antibodies, table 1b, were incubated with the samples for 1 hour at room temperature. Double labeling experiments were carried out by incubating cells in suitable combinations of primary antibodies, followed by non-cross reactive secondary antibodies. Undifferentiated hES cells and differentiated hBS cells were used in parallel as additional positive and negative controls.

TABLE 1
Antibodies
(a) Primary Antibodies

| Epitope | | | | | |
|---|---|---|---|---|---|
| name | Clone | Dilution | Description | Source | Staining |
| alpha- | C3 | 1:500 | Mouse | Sigma- | Endodermal |
| fetoprotein | | | IgG2a | Aldrich | |
| (AFP) | | | | | |
| alpha- | — | 1:250 | Mouse IgG1 | Chemicon | Human specific |
| human | | | | | |
| nuclei | | | | | |
| alpha- | ASM-1 | 1:200 | Mouse | Chemicon | Mesodermal |
| smooth | | | IgG2a | | |
| muscle | | | | | |
| actin | | | | | |
| (alpha- | | | | | |
| SMA) | | | | | |
| β-III-Tubulin | SDL.3D10 | 1:100 | Mouse | Sigma- | Ectodermal; |
| | | | IgG2b | Aldrich | neuronal precursor |
| | | | | | marker, also mature |
| | | | | | neurons. |
| PAN | Lu5 | 1:200 | Mouse IgG1 | Chemicon | All types of epithelia |
| cytokeratin | 131- | 1:100 | Mouse | Chemicon | Mesodermal |
| desmin | 15014 | | IgG1K | | |
| E-cadherin | HECD-1 | 1:500 | Mouse IgG1 | Zymed | Human epithelia |
| GFAP | — | 1:500 | Rabbit IgG | DAKO | Neuroectodermal; |
| | | | | | astrocytes |
| HNF3-β | M-20 | 1:500 | Goat IgG | Santa Cruz | Endodermal |
| nanog | — | 1:500 | Goat IgG | R & D | Undifferentiated |
| | | | | | cells |
| nestin | 25 | 1:200 | Mouse IgG1 | BD | Neuroectodermal |
| | | | | Biosciences | precursor cells |
| Oct-4 | C-10 | 1:200 | Mouse | Santa Cruz | Undifferentiated |
| | | | IgG2b | | cells |
| SSEA-1 | 480 | 1:200 | Mouse IgM | Santa Cruz | Early differentiated |
| | | | | | cells |
| SSEA-4 | 813-70 | 1:200 | Mouse IgG3 | Santa Cruz | Undifferentiated |
| | | | | | cells |
| TRA-1-60 | TRA-1-60 | 1:200 | Mouse IgM | Santa Cruz | Undifferentiated |
| | | | | | cells |
| vimentin | V9 | 1:300 | Mouse IgG1 | Chemicon | Mesodermal; |
| | | | | | mesenchymal cells, |
| | | | | | also in early |
| | | | | | differentiation |

TABLE 2
Secondary antibodies

| (b) Secondary Antibodies | | |
|---|---|---|
| Name | Dilution | Source |
| Goat anti mouse IgG Alexa | 1:1000 | Molecular Probes |
| Fluor₄₈₈ | | |
| Goat anti mouse IgG-FITC | 1:150 | Jackson ImmunoResearch |
| Goat anti mouse IgG_{2b} | 1:200 | Southern Biotech |
| Goat anti mouse IgM-FITC | 1:150 | Jackson ImmunoResearch |
| Donkey anti mouse IgG Alexa | 1:500 | Molecular Probes |
| Fluor₄₈₈ | | |
| Donkey anti rabbit IgG-FITC | 1:500 | Jackson ImmunoResearch |

As described above the hBS-MP cell lines could be maintained in a highly proliferative state in growth factor rich hBS-MP medium containing FBS and bFGF. Differentiation and growth arrest could be induced in vitro by switching the hBS-MP cell cultures to a serum-free hBS-MP medium without bFGF thus withdrawing mitogenic growth factors. Under those conditions proliferation of the hBS-MP cells nearly halted within 14 days while the cell morphology became significantly more spread out and flattened. Cytoskeletal elements became more pronounced in phase contrast microscopy.

### Proliferative State

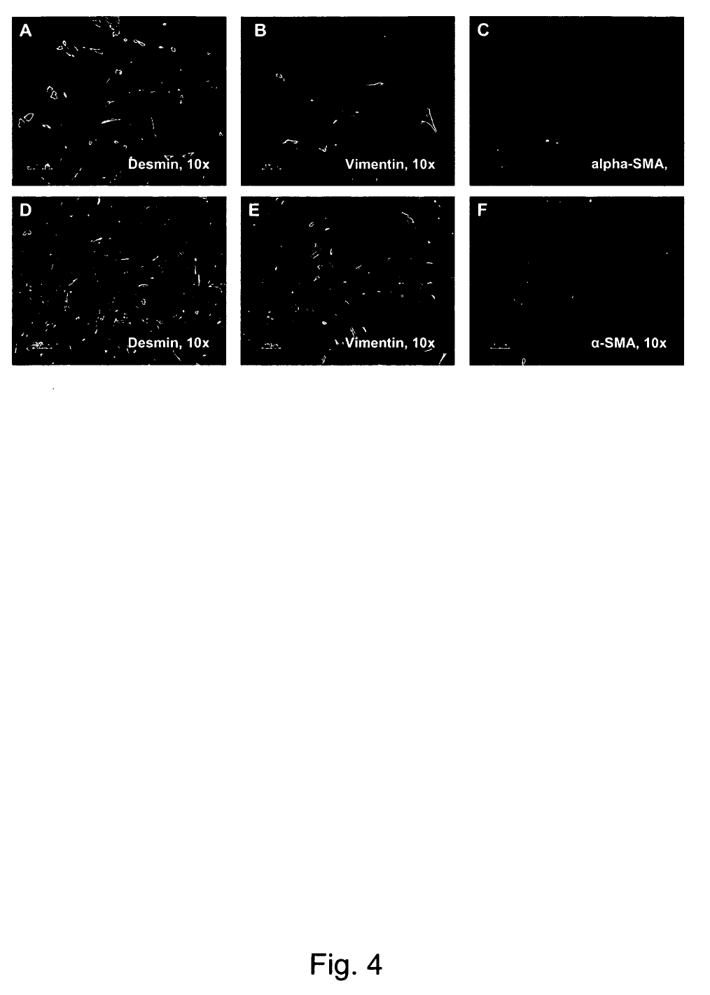

None of the markers typically found in undifferentiated hBS cells (Oct-4, Nanog, TRA 1-60, TRA 1-81, SSEA-3, SSEA-4) nor the early differentiation marker SSEA-1 could be detected in hBS-MP cell cultures in the proliferative state. None of the tested endodermal markers (HNF3β and AFP) were expressed. Neither the early neuroectodermal marker nestin nor the later markers GFAP and β-III-Tubulin could be detected. The epithelial markers E-cadherin and Pan-cytokeratin were negative. However, the early mesodermal markers desmin and vimentin were clearly expressed in almost all cells, while alpha-smooth muscle actin (ASMA) was expressed in individual cells in the proliferative state (FIG. 4 A-C).

Marker expression in the proliferative state as well as in the differentiated state was analyzed by immunocytochemistry. To evaluate the differentiation status of the cell lines, a set of markers commonly used in characterization of undifferentiated cells, was used. Furthermore a set of early markers for each germ layer (endoderm, mesoderm and ectoderm) were applied. Immunocytochemistry of hBS-MP cultures in the proliferative state and in the differentiated state (Table 1) showed complete absence of undifferentiated stem cell markers as well as ectodermal and endodermal markers while only mesodermal markers could be detected. Furthermore no epithelial markers were expressed.

TABLE 3
Immonocytochemistry results

| | | Differentiated |
|---|---|---|
| Immunocytochemical Marker | Proliferative State | state |
| Undifferentiated hES cells | | |
| SSEA-1 | Negative | Negative |
| SSEA-3 | Negative | Negative |
| SSEA-4 | Negative | Negative |
| TRA-1-60 | Negative | Negative |
| TRA-1-81 | Negative | Negative |
| Oct-4 | Negative | Negative |
| Nanog | Negative | Negative |
| Ectoderm | | |
| β_{III} tubulin | Negative | Negative |
| GFAP | Negative | Negative |
| Nestin | Negative | Negative |
| Endoderm | | |
| HNF3-β | Negative | Negative |
| alpha-fetoprotein (AFP) | Negative | Negative |
| Mesoderm | | |
| Vimentin | 100% | 100% |
| Desmin | >95% | 100% |
| alpha-smooth muscle actin (ASMA) | <5% | >90% |
| Epithelial | | |
| E-cadherin | Negative | Negative |
| Pan-cytokeratin | Negative | Negative |
| Human specific | | |
| alpha-human nuclei | 100% | 100% |

### Example 5

### Differentiation and Tissue Formation In Vitro

To remove mitotic growth factors in order to allow in vitro differentiation of hBS-MPs in monolayer culture, bFGF was withdrawn from the hBS-MP culture medium and FBS was replaced by SR (Serum replacement, Gibco, Invitrogen). The cells were then left to differentiate for 14 days in a humidified atmosphere at 37° C. and 5% CO₂. Culture medium was renewed every 2-3 days.

To evaluate the potential for tissue formation in-vitro, hBS-MPs were enzymatically removed from the culture dishes using TrypLE Select and transferred to 15 ml polypropylene centrifuge tubes (Falcon) at 3×10⁵ cells per tube. The cells were pelleted by centrifugation at 400 g for 5 min and subsequently maintained in suspension culture for 20 days in a humidified atmosphere at 37° C. and 5% CO₂. Culture medium was renewed every 2-3 days. Macroscopic examination revealed spherical pieces of tissue with a diameter of 0.5-1 mm. The tissue spheres were pliable (soft) but well coherent and could be handled with forceps without being damaged. The spheres were embedded in paraffin, cross-sectioned and stained for histology. Microscopic evaluation of the sections showed a homogenous tissue with spindle-shaped cells embedded in a large quantity of diffuse extracellular matrix (ECM) within the tissue sphere and a thin layer of elongated cells at the surface of the sphere. In general the tissue exhibited a high ECM to cell ratio.

Following differentiation in vitro the hBS-MP cultures were again evaluated using the above described panel of markers. Still no expression of the tested ectodermal and endodermal markers could be detected. No epithelial markers were expressed. The mesodermal markers desmin and vimentin were still expressed in almost all cells while interestingly ASMA expression had increased significantly so that now almost all cells were also ASMA positive (FIGS. 4 D-F).

### Example 6

### Dedifferentiation of hBS-MP Cells

hBS-MP cells were enzymatically removed from the culture dishes using TrypLE Select and seeded onto a growth-inactivated mouse embryonic fibroblast (mEF) feeder cell layer in VitroHES™ medium (Vitrolife AB, Kungsbacka, Sweden) supplemented with 4 ng/ml hbFGF. The co-cultures were incubated for 10 days in a humidified atmosphere at 37° C. and 5% CO₂. Culture medium was renewed every 2-3 days.

To evaluate whether hBS-MP cells could be steered back to a less differentiated state by the same signals that maintain pluripotency in hBS cells, proliferating hBS-MP cells were transferred to the traditional mEF feeder layer supported growth conditions for undifferentiated hBS cells.

Even under these conditions no evidence of de-differentiation could be found. The hBS-MP cells rapidly formed a confluent monolayer while maintaining a mesenchymal morphology. No cells with undifferentiated hBS cell morphology could be detected and immunocytochemical analysis with the undifferentiated hES cell markers Oct-4, SSEA-1 and TRA-1-60 was negative.

### Example 7

### In Vivo Differentiation by Transplantation of hBS-MP Cells and Subsequent Histological Analysis

For assessment of the safety and the in-vivo differentiation potential of the hBS-MPs approximately 200.000 hES-MP cells were mechanically scraped loose from the culture plate and surgically placed under the kidney capsule of 5 weeks old severe combined immuno-deficient mice (C.B-17/IcrCrl-Scid, Charles River Laboratories, Germany). The mice were sacrificed after 8 weeks and the kidneys were surgically removed. All animal studies had been reviewed and approved by the Institutional Animal Care and Use Committee at Göteborg University in accordance with the policy regarding the use and care of laboratory animals.

The tissue samples were fixed in 4% paraformaldehyde solution for 24 hours and embedded in paraffin. The tissue samples were sectioned at a thickness of 6-9 μm and were evaluated histologically following hematoxylin-eosin, alcain blue/van Giesson and Safranin orange staining. To confirm the human origin of the tissues developed under the fibrous capsule of the mouse kidneys a whole genome human DNA-FISH probe (Spectrum Red-labeled total human genomic DNA, Vysis Inc, Downers Grove, Ill., USA) was used to exclusively label human cell nuclei as described previously (Gertow et al., 2004).

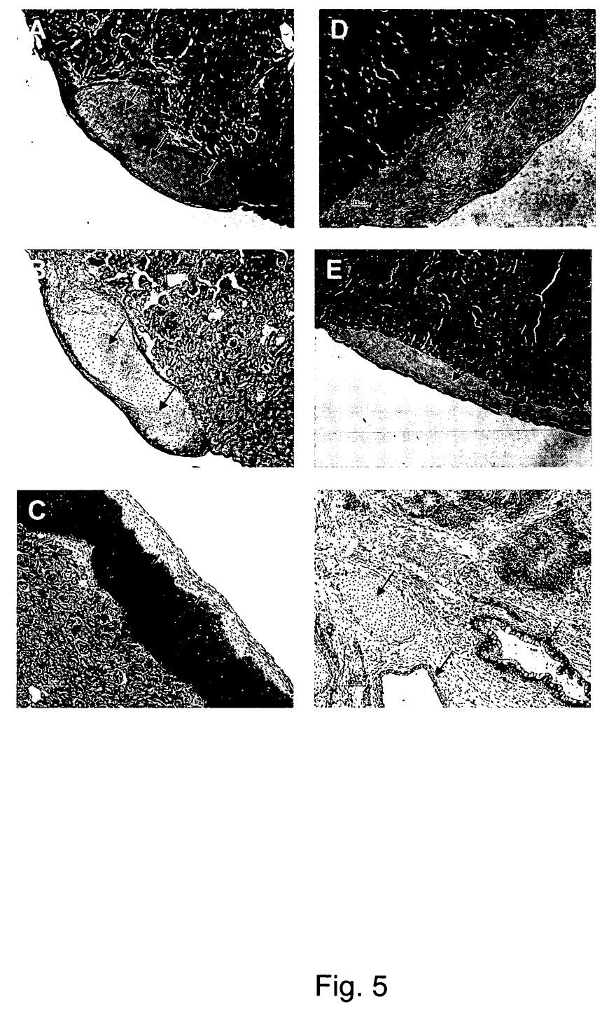

To examine the differentiation potential of the hBS-MPs in-vivo and to assess the risk of tumor formation after transplantation, clusters of hBS-MP cells were injected under the kidney capsule of SCID (severe combined immunodeficiency) mice. After 8 weeks the mice were sacrificed and the kidneys were surgically removed. In each case the injected hBS-MP cells had given rise to 1-2 mm oval tissue structures with sharp boundaries under the kidney capsule. The tissue formed by the hBS-MP cells was macroscopically clearly visible, but very small compared to the massive teratoma which develop after transplantation of small numbers of undifferentiated hES cells (Heins et al., 2004; Heins et al., 2005) and reach an average size of 1-2 cm within 8 weeks. Obviously growth of hBS-MP cells after transplantation is very limited. The samples were embedded, cross-sectioned and stained for further analysis. Histological evaluation of the tissue which had developed from the injected cells showed various tissues of embryonic/fetal mesenchymal origin either in pure form or in combinations. The observed tissues comprised soft and fibrous connective tissue, smooth muscle, immature tendon and hyaline cartilage. Presence of cartilage specific ECM was confirmed by Alcian Blue/Van Giesson and Safranin-orange staining. The human origin of the developed tissues was confirmed by whole genome human DNA-FISH (Data not shown). The injected hBS-MP cells had remained within a small, well limited area at the injection site and the developed tissue showed clearly defined boundaries towards the tissue of the mouse renal cortex. No sign of invasive growth into the surrounding tissue could be detected in serial cross-sections (FIGS. 5 (A)-(E)). Even at the microscopical level no indication of teratoma formation from the transplanted hBS-MP cells could be observed. All parental undifferentiated hBS cell lines on the contrary formed teratoma which comprised a mixture of tissues from all germ layers and showed invasive growth into the renal cortex (FIG. 5 F).

### Example 8

### Therapeutic Use in Human

The hBS-MP cells, no longer being undifferentiated hBS cells as shown by marker analysis and, accordingly, not giving rise to tumours in vivo, are still capable of proliferation and have a potential to differentiate into several mesenchymal cell types (including chondrocytes and connective tissue cells), and may therefore be suitable for treating disorders and diseases by reversing, inhibiting or preventing tissue damage. The hBS-MP cells may be used for treating disorders associated with, for example, necrotic, apoptotic, damaged, dysfunctional or morphologically abnormal connective tissue. In addition the cells may be used for reconstructive medicine and cell replacement therapy concerning fat, bone, muscle, tendon, and cartilage or for degenerative diseases, acute injuries and plastic surgery.

### Example 9

### Use of hBS-MP Cells for Measuring Cytotoxicity

hBS cells were dissociated into small cell aggregates and seeded into 96-well plates (Nunc, Kamstrupvej, Denmark) in 100 μl Test medium containing Knock Out DMEM supplemented with 20% Foetal bovine Serum (FBS), 1% penicillin-streptomycin, 1% Glutamax, 0.5 mmol/l β-mercaptoethanol and 1% non-essential amino acids (all Invitrogen). hBS-MP cells and hFF cells were dissociated into single cells and seeded into 96-well plates in 100 μl test medium.

After 24 hours the cytotoxicity test was started by adding 100 μl toxicity solution to the test wells that had twice the concentration as the required end concentration (day 0). Toxicity medium was changed on day 4 and 7 of the assay and on day 10 the plates were analysed measuring different endpoints for cytotoxicity, i.e. ATP content using Promega's CellTiterGlo Kit (Promega, Mannheim, Germany) according to the manufacturer's instructions and the reduction of Resazurin (Sigma, Stockholm, Sweden, CAS 62758-13-8) to the fluorescent Resofurin as described before (Evans et al., 2001). Both endpoints were analysed using a multi-detection reader (Fluostar Optima, BMG Labtech, Offenburg, Germany) measuring luminescence for the CellTiter Glo kit and fluorescence, at the wave lengths 530 nm (excitation) and 590 nm (emission) for the Resazurin assay.

The following chemicals were tested: 5-fluoro-uracil (5-FU) (Invivogen, Toulouse, France) as a positive control, sodium saccharin as a negative control, ATRA (all-trans-retinoic acid), and 13CRA (13-cis-retinoic acid) (all from Sigma). Saccharin was diluted in PBS (Invitrogen) to a concentration of 1 g/ml and stored in aliquots at 4° C. ATRA and 13CRA were dissolved in DMSO at a concentration of 0.1M and stored in aliquots at −20° C. The 5-FU solution (Invitrogen) and DMSO (Sigma), were directly diluted in the test medium. All chemicals were tested in a 3-fold dilution series with highest concentrations being: 27 μM for 5-FU, 1 mg/ml for Saccharin, 100 mM for ATRA and 13CRA. For ATRA and 13CRA the dilution series was performed in DMSO and the dilutions were then given to the test medium to obtain the final test concentrations. The DMSO concentration was for all tested concentrations 0.1%.

The IC50 values were obtained by fitting the four-parametric hill function to the data.

All cell types showed a toxic reaction to 5-FU and no toxic reaction to saccharin (data not shown).

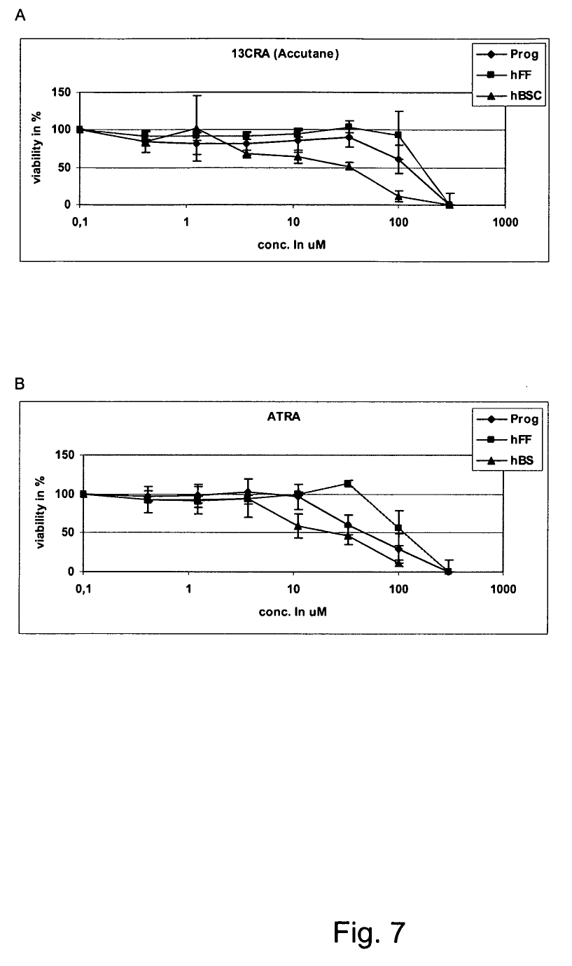

Comparisons of the IC50 values showed that the hBS cells and hBS-MP cells are much more sensitive towards the substances 5-FU, ATRA and 13CRA than the hFF cells, FIG. 7. The progenitor cells hence may represent one more easily cultured hBS cell type enabling larger scale culture with enzymatic passaging while maintaining a higher sensitivity to toxic substances.

TABLE 5
Ratios between the IC50 values measured using the Resazurin assay

| | hBS cells | hBS-MP cells | hFF cells |
|---|---|---|---|
| 5-FU | 1 | 3 | 7 |
| 13CRA | 1 | 4 | 10 |
| ATRA | 1 | 1 | 2 |

The initially undifferentiated hBS cells are equally sensitive as the progenitors or up to 4 times more sensitive. Moreover, the hBS system is between 2 and 10 times more efficient in predicting than the hFF test cells, while the progenitors are at least 2 times more efficient or sensitive compared to hFF.

### Example 10

### Characterization of hBS-MPs by Flow Cytometry Analysis

The phenotype of the hBS-MP SA002.0, SA002.5, SA167, and SA461 cell lines as well as undifferentiated hBS cells from the same cell lines were studied by flow cytometry analysis for a set of accepted markers for stem cells (CD117, CD133) and mesenchymal stem cells (CD105, CD166, CD10, CD13, Stro-1). Colonies of undifferentiated hBS cells cultured on a supporting feeder layer were dissected out and treated with 0.05% trypsin-0.53 mM EDTA (Gibco, Grand Island, N.Y.) to obtain a single cell suspension. The hBS-MP cells lines were expanded in monolayer as described above to 80% confluence, and were then harvested using 0.05% trypsin-0.53 mM EDTA. The cells were stained with CD105-PE (Ancell, Bayport, Minn.), CD166-FITC (Ancell), CD10-PE-Cy7 (Becton Dickinson, San Jose, Calif.), CD117-APC (Becton Dickinson), CD133-APC (Becton Dickinson), CD13-APC (Becton Dickinson), and Stro-1-PE (Santa Cruz Biotechnology, Santa Cruz, Calif.). At least 10.000 events were acquired for each sample using the FACS Aria flow cytometer and the FACSDiVa software (Becton Dickinson). The 488 nm argon ion laser was used to excite samples, with emission being measured using appropriate band pass filters. The cells were acquired and gated by forward (FSC) and side scatter (SSC) to exclude debris and cell aggregates. Dead cells were excluded by gating on FSC and SSC. To calculate the percentages of cells staining positive for each marker, a maximum of 2% positive cells stained with isotype control antibody was allowed.

### Results of Flow Cytometry Analysis

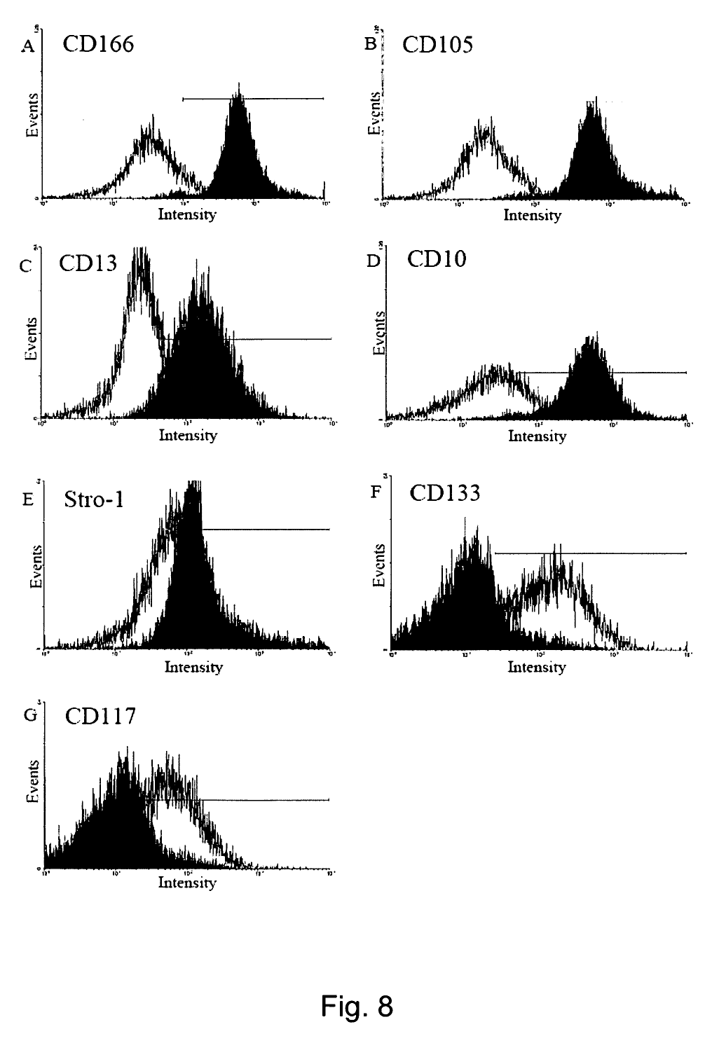

The hBS-MP cells lines consisted of a homogenous cell population were <99% of the cells expressed the mesenchymal stem cell markers CD166 and CD105 (FIGS. 8 A, B). In contrast, the undifferentiated hBS cell lines displayed a significantly lower expression of these markers (14.1% and 3.4% respectively) (FIGS. 8 A, B). The hBS-MP cell lines further had a high expression (<75%) of CD10, CD13, and Stro-1, while the undifferentiated hBS cells had a significantly lower expression (25%) of these surface markers (FIGS. 8 C-E). Only a small population of hES-MP cells were positive for the progenitor markers CD133 and CD117 (2.2% and 5.8% respectively) while a larger population of the undifferentiated hBS cells were positive for these markers (70.3% and 60.2% respectively) (FIGS. 8 F, G).

### Example 11

### Osteogenic Differentiation Model

Cubes of porous β-tricalcium phosphate ceramic (Biosorb, SBM, Lourdes, France) were rinsed in sterile water to remove ceramic dust and then dried for 2 h. The cubes were pre-coated by immersion into a 100 μg/mL solution of fibronectin (Sigma-Aldrich, Stockholm, Sweden) in capped polystyrene tubes. Air was withdrawn from the tubes through a tightly fitted cap with a 30-mL syringe fitted with a 20-gauge needle; the partial vacuum generated permitted the fibronectin solution to enter the pores within the cubes. The ceramic cubes were incubated in the fibronectin solution for 2 h at room temperature, transferred to a 12-wells culture dish, and then dried overnight in a laminar flow hood. The fibronectin-coated ceramic cubes were added to capped polystyrene tubes containing resuspended hES-MP cells from cell line 2.5 (2×106 cells were seeded per cube). Air was drawn from the tubes as for the fibronectin coating, facilitating exit of air bubbles to increase infiltration of cells into the cubes. The cell-loaded ceramic cubes was placed into an incubator at 37° C. for 2 h and were then cultured in DMEM-LG, 10% FCS, 80-μM ascorbic acid-2-phosphate (Wako Chemicals, USA) and 100-nM dexamethasone (Sigma-Aldrich). On Day 11, the osteogenic medium was further augmented with 2 mM β-glycerol phosphate (Sigma-Aldrich). After 6 weeks of differentiation, the ceramic cubes were fixed in Histofix™ (Histolab products AB, Gothenburg, Sweden), dehydrated with increasing concentrations of EtOH, decalcified, and embedded in paraffin. Five-micrometer sections were cut and placed onto silane-coated glass slides (Superfrost Plus, Menzel-Gläser, Germany). The sections were stained with Mallory Aniline Blue and Alcian Blue van Gieson stainings and were then observed with a light microscope (Nikon).

### Results

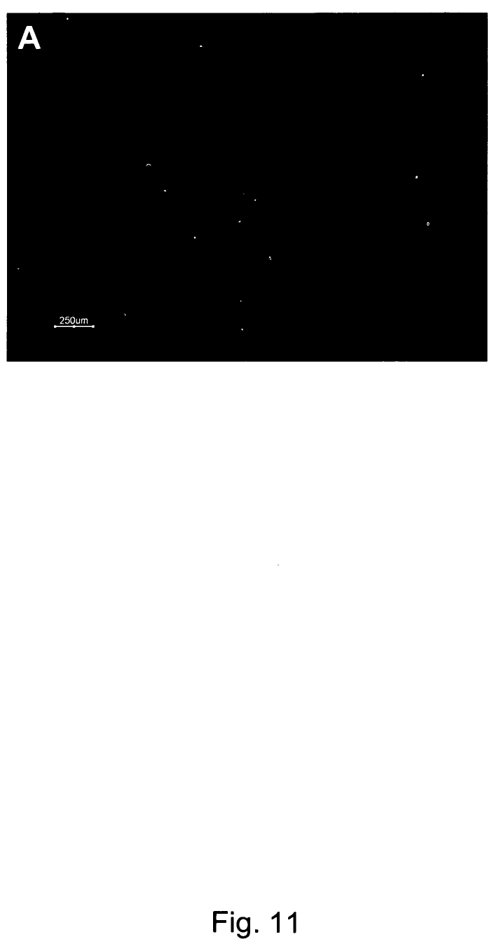

Examination of sections of the hBS-MP cell line 2.5 stained with Mallory Aniline Blue staining showed large orange to red areas corresponding to mineralized bone tissue. These areas had the same histological appearance as trabecular bone stained with Mallory Aniline Blue staining. These sections were further negative for Alcian Blue van Gieson staining, demonstrating the lack of sulphated proteoglycans characteristic for cartilage, FIG. 11.

### Example 12

### Mesenchymal Progenitor Cells Derived from hBS Cells can be Used as Feeders for hBS Cells

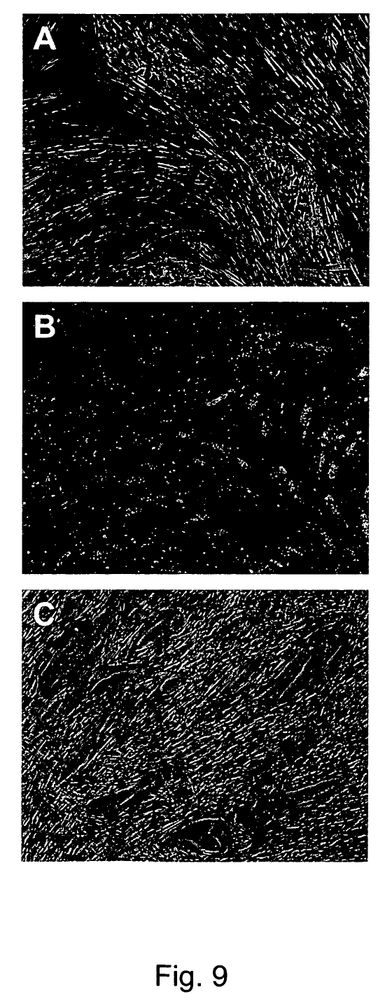

hBS-MPs in passage 4 and above have been tested for the ability to support hBS cells. Confluent or close to confluence T-25 or T-75 flasks containing hBS-MPs cells was treated with Mitomycin C for 2 hours in order to prevent the cells from further proliferation. The cells were dissociated and seeded into gelatin coated dishes, e.g. IVF dishes at a cell density from 70 000 cells per cm² to 140 000 cells per cm². The hBS-MPs cells were seeded in standard hBS-MPs medium, and 24 hours after seeding 100% of the medium was replaced with VitroHES medium plus 10 ng/ml bFGF. 2-5 days after seeding the hBS cells were transferred to the dishes. The hBS cells were either mechanical passaged or dissociated to single cells employing enzymes such as e.g. TrypLE Select. The hBS cells were then consecutively either enzymatic or mechanical passaged employing MP cells as feeders, FIG. 9. The hBS cells retained their morphological characteristics and expressed the expected stem cell markers such as e.g. Oct 4.

### Example 13

### Transfection of hBS-MPs

hBS cell cultures were enzymatically dissociated into single-cell suspension using TrypLE select (Gibco, Invitrogen). The cells were transfected using a transfection device giving stable integrated clones. The cells were counted and pelleted for 5 minutes, 400×g, 20° C. in a 15 ml centrifuge tube following re-suspension of 5×10⁵ cells in 10 μl resuspension buffer R (Digital Bio Technology) or 2×10⁶ cells in 110 μl kitV solution (AMAXA Biosystems). 2 μg plasmid DNA was added and the cell-DNA mix aspirated into a 10 μl MicroPorator pipette tip or transferred into a certified cuvette. The MicroPorator pipette was inserted into the MicroPorator pipette station and electroporated using 1350V, 30 ms, 1 pulse parameters.

After the pulse, cells were gently transferred into 0.1% porcine gelatin coated cell culture dishes (BD Biosciences, Bedford, Mass., USA) at 1×10⁴ cells per cm² in prewarmed hBS-MP medium consisting of DMEM (high glucose with glutamax, without pyruvate)+10% fetal bovine serum (FBS)+10 ng/ml human recombinant basic fibroblast growth factor (hrbFGF) (all from Gibco/Invitrogen).

FIG. 11 shows hBS-MPs expressing red fluorescent protein after transfection.

1. A novel mesenchymal human progenitor (hBS-MP) cell population negative for Sialic acid Neu5Gc and derived under xeno-free conditions from human blastocyst-derived stem (hBS) cells, wherein:
i) at least 80% of said cell population is negative for at least two markers reacting with undifferentiated hBS cells;
ii) at least 80% of said cell population is negative for at least one marker reacting with ectodermal lineage;
iii) at least 80% of said cell population is negative for at least one marker reacting with endodermal lineage; and
iv) at least 30% of said cell population is positive for at least one marker reacting with mesodermal lineage and the marker being selected from vimentin and desmin.

2. A cell population according to claim 1, wherein the markers reacting with undifferentiated hBS cells in step i) are selected from the group consisting of SSEA-3, SSEA-4, Tra1-60, Tra1-80, Oct-4, and Nanog.

3. A cell population according to claim 2, wherein as at least 80% of said cell population is negative for at least 3 of said five markers according to claim 2.

4. A cell population according to claim 1, wherein the markers reacting with the ectodermal lineage in step ii) are selected from the group consisting of beta-tubulin, GFAP, and nestin.

5. A cell population according to claim 4, wherein as at least 80% of said cell population is negative for at least two of said three markers according to claim 4.

6. A cell population according to claim 1, wherein the markers reacting with the endodermal lineage in step iii) are selected from the group consisting of HNF3-beta and AFP.

7. A cell population according to claim 6, wherein at least 80% of said cell population is negative for at least one of said two markers.

8. A cell population according claim 1, wherein at least 90% of said cell population is positive for at least one of the following markers reacting with the mesodermal lineage; vimentin and desmin.

9. A cell population according to claim 1, further characterized by;
v) less than 20% of said cell population is positive for the marker ASMA reacting with the mesodermal lineage.

10. A cell population according to claim 9, wherein less than 10% of said cell population is positive for the marker ASMA reacting with the mesodermal lineage.

11. A cell population according to claim 9, further characterized by;
vi) at least 80% of said cell population is negative for at least one marker reacting with the epithelial lineage.

12. A cell population according to claim 11, wherein the markers reacting with the epithelial lineage are selected from the group consisting of E-cadherin and pan-cytokeratin.

13. A cell population according to claim 1, having the potential to give rise to a progeny cell population, wherein at least 80% of said progeny cell population is positive for at least two of the following markers reacting with the mesodermal lineage; vimentin, desmin, and ASMA.

14. A cell population according to claim 1, wherein
i) at least 80% of said cell population express the mesenchymal stem cell markers CD166 and CD105,
ii) at least 60% of said cell population express the mesenchymal stem cell markers CD10, CD13 and Stro-1, and/or
iii) less than 10% of said cell population express the stem cell markers CD133 and CD117.

15. A cell population according to claim 14, wherein at least 90% of said cell population express the mesenchymal stem cell markers CD166 and CD105.

16. A cell population according to claim 14, wherein at least 75% of said cell population express the mesenchymal stem cell markers CD10, CD13 and Stro-1.

17. A cell population according to claim 1, wherein at least 80% of said cell population shows the following characteristic; a typical fibroblast-like morphology with elongated spindle-shaped cell morphology with branching pseudopodia and an elliptic nucleus.

18. A cell population according to claim 1 having the potential to form structures of one or more mesenchymal tissues and/or tissue derived from mesenchymal tissue in vitro and/or in vivo.

19. A cell population according to claim 18, wherein said structures resemble connective tissue.

20. A cell population according to claim 19, wherein said structures resemble cartilage, tendon and/or smooth muscle.

21. A cell population according to claim 1, wherein said cell population does not de-differentiate when transferring the hBS-MP cells back to a system for culturing undifferentiated hBS cells.

22. A cell population according to claim 1, wherein said cell population does not give rise to teratoma formation with all three germ layers present, when being engrafted into an immuno-deficient mouse.

23. (canceled)

24. (canceled)

25. A method to obtain an hBS-derived stem cell derived mesenchymal progenitor (hBS-MP) cell population without manual selection, said method comprising;
i) plating of undifferentiated hBS cells onto the surface;
ii) incubation of the plated cells for between 2 and 21 days to allow differentiation;
iii) enzymatic passaging to a second surface;
iv) repeating of step (iii) until a homogenously mesenchymal morphology is obtained; and
v) optionally, culturing the obtained hBS-MP cells.

26. A method according to claim 25, wherein the surface in step i) and/or step ii) is a tissue culture treated plastic or is a surface coated with a substance selected from mixed ECM extracts such as gelatin, Matrigel™, human placental matrix, or purified/synthetic ECM compounds.

27. A method according to claim 25, wherein the plated cells in step ii) are incubated for at least 5 days to allow differentiation until outgrowths of heterogeneous cell types occur.

28. A method according to claim 25, wherein the plated cells in step ii) are incubated for 5 to 7 days until outgrowths of heterogeneous cell types occur.

29. A method according to claim 25, wherein the cells after enzymatic treatment in the enzymatic passaging step ii) are in the form of a single cell suspension.

30. A method according to claim 25, wherein steps ii) and iii) lead to conditions which allow the selective survival and proliferation of hBS-MPs to maintain already formed hBS-MP cells to proliferate, without significant differentiation.

31. A method according to claim 30, wherein the selection pressure applied avoids additional selection of hBS-MP cells in step iii) and/or step iv).

32. A method according to claim 25, wherein the enzymes used in step iii) are selected from the group consisting of trypsin, TrypLE™ select, accutase alone or in combination with Ca-chelator.

33. A method according to claim 25, wherein the culture medium used is chosen from a group comprising, but not limited to, Vitrohes™, Vitrohes™ with bFGF, human recombinant FGF and/or FBS, and hBS-MP cell medium, a mammalian cell culture medium in combination with serum.

34. A method according to claim 33, wherein the concentration of bFGF or human recombinant FGF is in the range of 0.1-100 ng/ml.

35. A method according to claim 33, wherein the concentration of FBS is in the range 1-40.

36. A method according to claim 25, wherein said cell population can be cultured without any feeder cells or conditioned medium present.

37. A method according to claim 25, wherein said cell population can be cultured directly on plastic.

38. A method according to claim 25, wherein said cell population can be passaged at a split ratio in step v) between 1:5 and 1:40.

39. A method according claim 25, wherein all reagents used are xeno-free in order to obtain xeno-free hBS-MP cells.

40. A drug discovery process comprising utilizing the cell population of claim 1.

41. A process for studying drugs with potential effect on mesenchymal cell types comprising utilizing the cell population of claim 1.

42. A process utilizing feeder cells wherein the cell population of claim 1 provides said feeder cells which are utilized.

43. A process for studying genesis of mesenchymal tissues wherein the cell population of claim 1 serves the role of an in vitro model.

44. A process for studying human degenerative disorders comprising utilizing the cell population of claim 1.

45. A process for in vitro toxicity testing comprising utilizing the cell population of claim 1.

46. A process for the detection and/or prediction of in vitro toxicity in the human species, wherein the cell population of claim 1 is utilized and the assay enables novel detection of toxicity for a substance and/or more efficiently detects toxicity compared to non-human assays or assays based on adult human cell types.

47. The process according to claim 46 in in vitro toxicity assays, wherein the endpoint is cytotoxic.

48. The process according to claim 47, wherein the toxicity is visualized by resazurin conversion.

49. The process according to claim 48, wherein the toxicity is visualized by ATP content analysis.

50. A process for regenerative medicine comprising utilizing the cell population of claim 1.

51. (canceled)

52. A process for the manufacture of a medicinal product for the prevention and/or treatment of pathologies and/or diseases caused by tissue degeneration comprising utilizing the cell population of claim 1.

53. A process for the manufacture of a medicinal product for the treatment of connective tissue disorders comprising utilizing the cell population of claim 1.

54. (canceled)

55. A process for obtaining mesodermal cell types comprising utilizing the cell population of claim 1.

56. A process for studying maturation towards connective tissue cells comprising utilizing the cell population of claim 1.

57. A process for obtaining cardiomyocytes comprising utilizing the cell population of claim 1.

58. A kit for deriving and/or culturing hBS-MP cells as defined in claim 1 comprising:
i) undifferentiated hBS cells;
ii) one or more culture media, chosen from a group comprising, but not limited to, Vitrohes™, Vitrohes™ with bFGF, hBS-MP cell medium;
iii) one or more suitable enzymes, and
iii) optionally, an instruction for use.

59. A kit for regenerative medicine said kit comprising:
i) hBS-MP cells as defined in claim 1;
ii) optionally, factors for driving differentiation in vitro and/or in vivo; and
iii) tools for administration of the cells to a patient or cells in an form suitable for administration.

60. A progenitor-cell based kit for detecting toxicity in human, said kit comprising:
i) hBS-MP cells as defined in claim 1;
ii) optionally, positive and negative control substances;
iii) one or more reagents for detecting and/or measuring cytotoxicity; and
iv) optionally an instruction for use
